SEQUENCE LISTING (47 sequences)

SEQ ID NO: 1 - DNA, 1550 bp, Mus musculus
atgaggaaccctgggggcccaggttgggcatcaaaaaggcccctgcagaagaagcagaacacagcctgcctctgtgcccagcagccagccagacactttgtaccggctcccttcaactcgtccaggcagggcaagaacacggcccagccgacagagccctcgctctccagcgtgattgcgcccacactcttctgtgcgtttctttacttggcttgtgttactgctgaacttccagaggtgagcagaaggatggccaccagcggagtcagaagcaaggaaggacgccgggagcatgccttcgtcccagaacctttcactggtactaacttagctcccagcctttggctgcaccgctttgaagtcattgatgacctcaaccattgggatcatgccaccaaactgaggttcctgaaagagtcgctcaagggagatgccctggatgtctacaatggactcagttcccaggcccagggcgatttcagttttgtgaagcaagccctcctgagggcctttggggcccctggggaggccttcagtgagcccgaagagattttgtttgccaacagcatgggtaagggctactaccttaaagggaaggttggccatgtgcctgtgagattcctggtggactctggggctcaggtgtctgtggttcaccccgccttatgggaggaggtcactgatggtgacctggatactcttcgtccttttaacaatgtggtcaaagtggccaatggggcagagatgaagatcttgggtgtgtgggacacagaaattagcctgggcaagacaaagctgaaggccgagtttctggtggccaacgccagcgcagaagaggctattattggcacagacgtcttgcaggaccacaatgccgtgctggacttcgaacaccgcacctgcaccctgaaggggaagaagttccgcctgctccctgtcgggagctccttggaggatgagtttgacctggagcttattgaggaagaggaggggtcttctgcaccggagggctcccactaagaaaccccatttcttgttcccagcattggtagggggactttgtgttggggggagcagatgtcctggggggtatcatccggcctagccagtctttacaccggttctcagtttccctccttctacaggggccttgctttgcctttgtttggggagggaggccagcttggtggcctaaagcagtgtccccaaggtctgcaaagacttccaaggctggcaggagcttctgaggaagccaggaatgtcaatcttgagagaggacccttttagatcccctgaagtatggctcagtcactttcacgtccccaagcctgctgagctgagcctggtcttggctaagaccctcacaatccagatgcttggaggagactggcagctgctctgggagtcctccctgagtcctcccacctgcacaaggatgctccctcctgtcctgtcacttgccttgaatctcatggagcctgtatcaataattcaattatttcaaaacaccaataaagatctgttcatggaaaaaaaaaaaaaaaaaaaaa

SEQ ID NO: 2 - protein, 339 aa, Mus musculus
MRNPGGPGWASKRPLQKKQNTACLCAQQPARHFVPAPFNSSRQGKNTAQPTEPSLSSVIAPTLFCAFLYLACVTAELPEVSRRMATSGVRSKEGRREHAFVPEPFTGTNLAPSLWLHRFEVIDDLNHWDHATKLRFLKESLKGDALDVYNGLSSQAQGDFSFVKQALLRAFGAPGEAFSEPEEILFANSMGKGYYLKGKVGHVPVRFLVDSGAQVSVVHPALWEEVTDGDLDTLRPFNNVVKVANGAEMKILGVWDTEISLGKTKLKAEFLVANASAEEAIIGTDVLQDHNAVLDFEHRTCTLKGKKFRLLPVGSSLEDEFDLELIEEEEGSSAPEGSH

SEQ ID NO: 3 - DNA, 2216 bp, Homo sapiens
gcagcagttgagtccatgtaagatgattgtgcacgtgtgtgtgtgtgtgtgtgtgtgtgtgtgtgtcctcactcacttaaaaaagaccatttctgtgcattctttatgctggaaccctcctctgcctccctctccaggaaatcccataggatcccatacctgctttagaatgtgtgtgagacacttcccagtgcctggcatgtggtaggagctcagtacatgtaaactgttttcctttgaccactcaaagtagtcacttgttggggaggccaatggcggcttccccggtctctgagctgatgggcgtctggtccctgcctccccttccttcactggctgatgacaaatacctgaatgtcatcacatcctccgtgtcctcatctgttattctggaacacaggccagggagggtacagggtagtcccccttcaacagaggctggcctttcttgggatgtgggctggctggtgttggtccaaggatggatgcagagggtgaggcacccatcctgctagtccggccggatgctggcaggagggcggggtgaggaggggcggagcttccagaacaaaggagaatggggagcccaggggccagcctaggcatcaaaaaggctctgcagagtgaacaggccacagcactgcctgcctctgccccagcagtcagccagccgaccgcgcctgctccctcctgcttgcccaaggccgggcaagtcatccccactctgcttcgagaggccccgttttccagcgtgattgcgccgacactgctctgtgggtttctcttcttggcgtgggttgctgctgaggttccagaggagagcagcaggatggccgggagcggagccaggagtgaggaaggccgccggcagcatgccttcgtcccggaaccttttgatggggccaatgtcgtcccaaacctctggctgcacagctttgaagtcatcaatgacctcaaccattgggaccatatcaccaagctaaggttcctgaaagagtccctcagaggagaggccctgggtgtctacaataggctcagtccccaggaccagggagactatgggactgtgaaagaggccctcctgaaggcctttggggtccctggggctgcccccagccacctgcccaaagagatcgtctttgccaacagcatgggtaagggctactatctcaaggggaagattggcaaagtgcccgtgaggttcctggtggactctggggcccaggtctctgtggtccacccaaacttgtgggaggaggtcactgatggcgatctggacaccctgcagccctttgagaatgtggtaaaggtggccaatggtgctgaaatgaagatcctgggtgtctgggatacagcggtgtccctaggcaagctgaagctgaaggcacagttcctagtggccaatgcgagtgccgaggaagccatcattggcactgatgtgctccaggaccacaatgctatcctggactttgagcaccgcacatgcaccctgaaagggaagaagtttcgccttctgcctgtgggagggtccctggaagatgagtttgacctggagctcatagaggaggacccctcctcagaagaagggcggcaggagctatcccactgagaagccaccttttctttaacctcctaaatattggtgggaagacccaccgctgtggggggggttgcatatcctcatgggggtcactgggcttggccagtctgcttatcaactcttgctcttctctcccctttgcctccctctgcaggggccttaatctgcccctggtaggggaggcttccactgaacaggcacaggtgagggagagcaggctggcttagagggacagggtccccatggtcatcaagctgctgttgatgacaaagactcaaaggctggaagagctcccaaggaagctagaaatgcttgtctttgaaagaactgtgggaccccttcagattccctgaggtatggcttggtcactctcaggtcctcaaagcctgtcttagttgggctgggtcctagctgcagggtctttgtgagggtcacagttgctctgggacacctccctgaagagcctttccacctgtacaatcgtattttctttctgtcatttgctttgaagcccattgtgccttatgccaataattcaattgctgcaaacaccaataaagattgattcatggaaaaaaaaaaaaaaaaaaaaaaaaaaaaaaaaaaaaaaaaaaaa

SEQ ID NO: 4 - protein, 343 aa, Homo sapiens
MGSPGASLGIKKALQSEQATALPASAPAVSQPTAPAPSCLPKAGQVIPTLLREAPFSSVIAPTLLCGFLFLAWVAAEVPEESSRMAGSGARSEEGRRQHAFVPEPFDGANVVPNLWLHSFEVINDLNHWDHITKLRFLKESLRGEALGVYNRLSPQDQGDYGTVKEALLKAFGVPGAAPSHLPKEIVFANSMGKGYYLKGKIGKVPVRFLVDSGAQVSVVHPNLWEEVTDGDLDTLQPFENVVKVANGAEMKILGVWDTAVSLGKLKLKAQFLVANASAEEAIIGTDVLQDHNAILDFEHRTCTLKGKKFRLLPVGGSLEDEFDLELIEEDPSSEEGRQELSH

SEQ ID NO: 5 - protein, 256 aa, Mus musculus
MATSGVRSKEGRREHAFVPEPFTGTNLAPSLWLHRFEVIDDLNHWDHATKLRFLKESLKGDALDVYNGLSSQAQGDFSFVKQALLRAFGAPGEAFSEPEEILFANSMGKGYYLKGKVGHVPVRFLVDSGAQVSVVHPALWEEVTDGDLDTLRPFNNVVKVANGAEMKILGVWDTEISLGKTKLKAEFLVANASAEEAIIGTDVLQDHNAVLDFEHRTCTLKGKKFRLLPVGSSLEDEFDLELIEEEEGSSAPEGSH

SEQ ID NO: 6 - protein, 259 aa, Homo sapiens
MAGSGARSEEGRRQHAFVPEPFDGANVVPNLWLHSFEVINDLNHWDHITKLRFLKESLRGEALGVYNRLSPQDQGDYGTVKEALLKAFGVPGAAPSHLPKEIVFANSMGKGYYLKGKIGKVPVRFLVDSGAQVSVVHPNLWEEVTDGDLDTLQPFENVVKVANGAEMKILGVWDTAVSLGKLKLKAQFLVANASAEEAIIGTDVLQDHNAILDFEHRTCTLKGKKFRLLPVGGSLEDEFDLELIEEDPSSEEGRQELSH

SEQ ID NO: 7 - DNA, 21 bp, Artificial Sequence
  Primer
ccacctgcccaaagagatcgt

SEQ ID NO: 8 - DNA, 22 bp, Artificial Sequence
  Primer
atgggaggaggtcactgatggt

SEQ ID NO: 9 - DNA, 22 bp, Artificial Sequence
  Primer
ggctctccagaacatcatccct

SEQ ID NO: 10 - DNA, 24 bp, Artificial Sequence
  Primer
gactgccattacctgtgccttaga

SEQ ID NO: 11 - DNA, 20 bp, Artificial Sequence
  Primer
ctccctgcggcggcttgaac

SEQ ID NO: 12 - DNA, 26 bp, Artificial Sequence
  Primer
gggtctcatccacacagcttattagg

SEQ ID NO: 13 - DNA, 25 bp, Artificial Sequence
  Primer
caggactgaaagacttgctcgagat

SEQ ID NO: 14 - DNA, 21 bp, Artificial Sequence
  Primer
atcgccatcagtgacctcctc

SEQ ID NO: 15 - DNA, 22 bp, Artificial Sequence
  Primer
ctcggccttcagctttgtcttg

SEQ ID NO: 16 - DNA, 22 bp, Artificial Sequence
  Primer
acgcctgcttcaccaccttctt

SEQ ID NO: 17 - DNA, 24 bp, Artificial Sequence
  Primer
atgaactggtacacgaaatgcctt

SEQ ID NO: 18 - DNA, 26 bp, Artificial Sequence
  Primer
cctaataagctgtgtggatgagaccc

SEQ ID NO: 19 - DNA, 21 bp, Artificial Sequence
  Primer
ctcagcaccatcagcaccacc

SEQ ID NO: 20 - DNA, 21 bp, Artificial Sequence
  Primer
ggctcttcaggctgggatgct

SEQ ID NO: 21 - DNA, 26 bp, Artificial Sequence
  Primer
cagcaggtcagcaaagaacttatagc

SEQ ID NO: 22 - DNA, 20 bp, Artificial Sequence
  Primer
ggcattaaagcagcgtatcc

SEQ ID NO: 23 - DNA, 20 bp, Artificial Sequence
  Primer
aagggagctgcagtggagta

SEQ ID NO: 24 - DNA, 20 bp, Artificial Sequence
  Primer
aaccagaagtggcacctgac

SEQ ID NO: 25 - DNA, 20 bp, Artificial Sequence
  Primer
ccgaaaatctgtgggaagtc

SEQ ID NO: 26 - DNA, 20 bp, Artificial Sequence
  Primer
ccaagttctattccatcaga

SEQ ID NO: 27 - DNA, 29 bp, Artificial Sequence
  Primer
agcttgagcccttgagcccacagatttac

SEQ ID NO: 28 - DNA, 30 bp, Artificial Sequence
  Primer
actatctatctagctatctgcatgtctatc

SEQ ID NO: 29 - DNA, 26 bp, Artificial Sequence
  Primer
gaggagacggtgaccgtggtccctgc

SEQ ID NO: 30 - DNA, 20 bp, Artificial Sequence
  Primer
ctactggatgcactgggtga

SEQ ID NO: 31 - DNA, 26 bp, Artificial Sequence
  Primer
ggaccagggggctcaggtcactcagg

SEQ ID NO: 32 - DNA, 21 bp, Artificial Sequence
  Primer
ttggccccagtagtcaaagta

SEQ ID NO: 33 - DNA, 20 bp, Artificial Sequence
  Primer
acgtccagacacagcatagg

SEQ ID NO: 34 - DNA, 19 bp, Artificial Sequence
  Primer
ggttttatctgtgcagccc

SEQ ID NO: 35 - DNA, 21 bp, Artificial Sequence
  Primer
gaggtccaagagactttctgg

SEQ ID NO: 36 - DNA, 20 bp, Artificial Sequence
  Primer
ctctacacccccaacacacc

SEQ ID NO: 37 - DNA, 20 bp, Artificial Sequence
  Primer
tcgccttcttgacgagttct

SEQ ID NO: 38 - DNA, 20 bp, Artificial Sequence
  Primer
agtgagcgagcacagacaag

SEQ ID NO: 39 - DNA, 20 bp, Artificial Sequence
  Primer
ggcggatttctgagttggag

SEQ ID NO: 40 - DNA, 20 bp, Artificial Sequence
  Primer
ctgggctatttgtatcttca

SEQ ID NO: 41 - DNA, 20 bp, Artificial Sequence
  Primer
gcagcgcatcgccttctatc

SEQ ID NO: 42 - DNA, 20 bp, Artificial Sequence
  Primer
cacacctgctggtccaagtc

SEQ ID NO: 43 - DNA, 21 bp, Artificial Sequence
  Primer
ctgggagttcaaggttggtct

SEQ ID NO: 44 - DNA, 21 bp, Artificial Sequence
  Primer
ccacagtctcaagtctttcct

SEQ ID NO: 45 - DNA, 21 bp, Artificial Sequence
  Primer
gcaggagagggctgggaaaaa

SEQ ID NO: 46 - DNA, 31 bp, Artificial Sequence
  Primer
taccgtccgccgttttctcgcgttgttcacc

SEQ ID NO: 47 - DNA, 30 bp, Artificial Sequence
  Primer
aaacggcggacggtaccagccaacttccat

(19) United States
(12) Patent Application Publication
(10) Pub. No.: US 2020/0355684 A1
(43) Pub. Date: Nov. 12, 2020

(54) ASPRV1 AS A NEUTROPHIL-SPECIFIC MARKER AND THERAPEUTIC TARGET FOR INFLAMMATORY DISEASES

(71) Applicant: Université Laval, Québec (CA)

(72) Inventor: Luc VALLIÈRES, Québec (CA)

(21) Appl. No.: 16/622,367

(22) PCT Filed: Jul. 9, 2018
(86) PCT No.: PCT/CA2018/000140

§ 371 Date: Dec. 13, 2019

Related U.S. Application Data
(60) Provisional application No. 62/533,398, filed on Jul. 17, 2017.

(51) Int. Cl. G01N 33/573; C07K 16/40; A61P 37/06; C12Q 1/6881; C12Q 1/6883; G01N 33/569; A61K 47/68; G01N 33/68; A61P 25/28

(52) U.S. Cl. CPC G01N 33/573; G01N 2800/285; A61P 37/06; C12Q 1/6881; C12Q 1/6883; G01N 33/56972; A61K 47/6871; G01N 33/6896; A61P 25/28; C07K 2317/76; C07K 2317/34; C12Q 2600/158; G01N 2333/96472; G01N 2500/02; C07K 16/40

(57) ABSTRACT

Neutrophils contribute to demyelinating autoimmune diseases, yet their phenotype and functions have been elusive to date. The present application shows that ICAM1 surface expression distinguishes extra- from intravascular neutrophils. Transcriptomic analysis of these two subpopulations indicated that neutrophils, once extravasated, acquire macrophage-like properties, including the potential for immunostimulation and MHC class II-mediated antigen presentation. Further, the present application shows that aspartic retroviral-like protease ASPRV1 is specifically expressed by neutrophils in the mouse and human immune system, and correlates with CNS neutrophil infiltration in EAE, multiple sclerosis and neuromyelitis optica.

### PRIORITY CLAIM

This patent application is a national stage filing under 35 U.S.C. § 371 of international application No. PCT/CA2018/000140 filed on Jul. 9, 2018 which claimed priority to U.S. provisional application No. 62/533,398 filed on Jul. 17, 2017. The entire contents of which is incorporated herein by reference.

### SEQUENCE LISTING

In accordance with Section 1.1 of the Legal Framework for EFS-Web, a Sequence Listing in the form of an ASCII text file is submitted via EFS-Web entitled “200325_PCTCA2018000140_ST25.txt” created on Mar. 25, 2020 of 23,196 bytes) and is incorporated herein by reference.

### FIELD OF THE INVENTION

The present invention relates to aspartic peptidase retroviral-like 1 (ASPRV1, also known as SASPase) as a neutrophil-specific marker and therapeutic target for inflammatory diseases. The present invention also relates to molecules that specifically bind to ASPRV1 and that may serve as markers of neutrophils or as inhibitors of ASPRV1 for the treatment of diseases-related to or caused by neutrophils.

### BACKGROUND

People with demyelinating autoimmune diseases, such as multiple sclerosis (MS), neuromyelitis optica spectrum disorder (NMOSD) and acute disseminated encephalomyelitis (ADEM), have a corrupted immune system that destroys the myelin sheath of neurons in the central nervous system (CNS). The root causes of these diseases remain unknown, but the underlying molecular and cellular mechanisms are fairly well comprehended, owing to the animal model experimental autoimmune encephalomyelitis (EAE). EAE can be induced in mice by immunization with different myelin antigens (e.g. myelin oligodendrocyte glycoprotein [MOG])¹. Similarly to its human counterparts, EAE can comprise five phases: 1) a preclinical induction phase—in which CD4⁺ T helper (Tₕ) cells are primed in lymphoid organs by antigen-presenting cells (e.g. dendritic cells [DCs], B cells) and traffic towards the CNS²; 2) an onset phase—in which Tₕ cells are licensed at the CNS interface to enter the parenchyma and initiate inflammation³; 3) an acute attack phase—in which myeloid cells (e.g. macrophages) execute effector functions resulting in demyelination⁴; 4) a recovery phase—in which immunoregulatory cells act to resolve inflammation either partially or completely⁵; and 5) a chronic phase—in which corrupted immune cells succeed in perpetuating the inflammation. The mechanism responsible for the progression of acute to chronic inflammation is unknown, yet critical for understanding and treating autoimmune diseases.

The past decade of research has unmasked a new player in autoimmune demyelination: the neutrophil granulocyte. As discussed in a recent review⁶, this type of myeloid cell is observed in the CNS during NMOSD, ADEM and severe forms of MS. In the most common forms of MS, neutrophils are typically absent from brains taken at autopsy, usually many years after diagnosis. However, one study reports their presence in the cerebrospinal fluid of early-diagnosed MS patients⁷, and several indirect observations link neutrophils to the most common forms of MS⁶. In EAE pathogenesis, the importance of neutrophils is clear: the disease can be blocked or attenuated by depleting neutrophils or molecules involved in their recruitment⁶. Yet, their exact functions remain elusive, in part because their phenotype has not yet been characterized, and because there is a paucity of biomarkers and genetic deletion tools for neutrophil assessment.

In the present study, we show that neutrophils that infiltrate the spinal cord parenchyma during EAE, but not intravascular neutrophils that crawl on the luminal endothelial surface, bear on their surface the adhesion molecule ICAM1(CD54). Using this marker, we isolated these two neutrophil populations and compared their transcriptomes in order to gain insights into their properties. The results suggest that neutrophils, in EAE, exert macrophage-like functions as well as a novel effect via the enzyme aspartic peptidase retroviral-like 1 (ASPRV1, also known as SASPase). ASPRV1 is synthesized as a zymogen that contains a putative transmembrane domain and a conserved catalytic domain with a key aspartic acid residue⁸⁻¹⁰. This zymogen can undergo auto-cleavage under slightly acidic conditions, releasing the catalytic domain that homodimerizes to form an active protease^{8,9}. ASPRV1 has so far only been detected in stratified epithelia where it cleaves its only known substrate, profilaggrin^{9,11,12}. Its knockdown or overexpression causes no major physiological defect (although the skin of adult ASPRV1-deficient mice shows fine wrinkles and reduced hydration)⁹,11,12. Here we demonstrate that ASPRV1 is: 1) only expressed by neutrophils in the immune and nervous systems both in mouse and human; and 2) essential for the progression of acute to chronic inflammation, specifically when EAE is induced with a new MOG antigen that involves, like in MS¹³ and contrary to the traditional MOG₃₅₋₅₅ peptide⁴, a deleterious action of B cells.

U.S. Pat. No. 7,834,238 to Matsui et al. describes knockout mouse in which the gene encoding SASPase has been deleted. The amino acid sequence of the mouse ASPRV1 protein is set forth in SEQ ID NO.:2 The active fragment of ASPRV1 corresponding to amino acid position 84 to 339 of SEQ ID NO.:2 is produced by auto-processing and the protease activity appears to be located within amino acid 189 to 324 of SEQ ID NO.:2.

Bernard et al., report that indinavir (a potent HIV protease inhibitor) had a significant inhibitory effect on recombinant human ASPRV1 autoactivation, whereas norvir, ritonavir, amprenavir, pepstatine, saquinavir did not 8. However it is possible that other retroviral protease inhibitors have an inhibitory effect on ASPRV1. For example, the literature reports Indinavir analogues (e.g., CH05-0, CH05-10, XN1336-27, XN1336-51, XN1336-52; 807-29-4) and it is possible that such analogues have similar inhibitory properties towards ASPRV1 (You, J et al. Cancer Science, 101, pp. 2644-2651, 2010; King, N. et al., Protein Science (2002), 11:418-429).

### SUMMARY OF THE INVENTION

The present invention relates to ASPRV1 as a neutrophil-specific marker and therapeutic target for inflammatory diseases. The present invention also relates to molecules that specifically bind to ASPRV1 which may serve as markers of neutrophils or as inhibitors of ASPRV1 for the treatment of diseases-related to or caused by neutrophils.

More particularly, methods for identifying or detecting neutrophils are provided, which rely on the detection of the aspartic peptidase retroviral-like 1(ASPRV1) protein or nucleic acids encoding ASPRV1 such as mRNA and cDNA. The method may be particularly applied for detecting ASPRV1 or an ASPRV1 fragment located within a cell or a cell compartment. An increased level of ASPRV1 or neutrophils expressing ASPRV1 may be indicative of an inflammatory or autoimmune disease. As such, inhibitors of neutrophils or specific inhibitors of ASPRV1 may be administered to a mammal where such an increase is observed.

Also encompassed by the present invention, are methods for targeting neutrophils in mammals by administration of compounds that specifically bind to the ASPRV1 protein (or to a fragment thereof) or to a nucleic acid encoding the ASPRV1 protein. For example, an anti-ASPRV1 antibody or an antigen-binding fragment thereof may be particularly suitable for such purpose especially when provided in the form of pharmaceutical compositions.

The present invention also relates to methods of treatment of inflammatory or autoimmune disorders associated with neutrophils, involving administration of compounds that specifically target ASPRV1 such as an antibody or antigen-binding fragment thereof.

The present invention also relates to an antibody or antigen binding fragment thereof that binds to ASPRV1. More particularly, the antibody or antigen binding fragment thereof may bind to a short form of ASPRV1. In an exemplary embodiment the short form of ASPRV1 may have an amino acid sequence as set forth in SEQ ID NO.:5 or in SEQ ID NO.:6.

The present invention more particularly relates to a method of treating a mammal having a disease associated with neutrophils, which may comprise administering the antibody or an antigen binding fragment thereof of the present invention. More particularly, the present invention relates to method of treating human with an antibody or antigen binding fragment thereof that may bind to human ASPRV1 (SEQ ID NO.:4) or to a human ASPRV1 fragment thereof, including, for example, the short form of human ASPRV1 set forth in SEQ ID NO.:6.

In accordance with the present invention, the disease may be an inflammatory or autoimmune disorder.

Also in accordance with the present invention, the ASPRV1 fragment thereof may be an ASPRV1 active fragment thereof. Further in accordance with the present invention, the ASPRV1 protein may be a short form of ASPRV1.

Inhibitors of ASPRV1 may be identified by methods comprising independently contacting an ASPRV1 protein or an active fragment thereof or a cell expressing the ASPRV1 protein or the active fragment thereof with a series of putative inhibitors the structure of which is identifiable and identifying those that inhibits the enzymatic activity of ASPRV1, the dimerization of ASPRV1, auto-cleavage of ASPRV1 or that binds the same site as indinavir or that competes with indinavir. A collection of antibodies or antigen binding fragments obtained from an animal immunized with ASPRV1 may be screened and antibodies having inhibitory effects selected. Alternatively, a library of small molecules, peptides, peptidomimetics or else may be screened in order to identify suitable compounds having inhibitory effects towards ASPRV1.

It is to be understood herein that the present invention is particularly applicable to human ASPRV1.

### BRIEF DESCRIPTION OF DRAWINGS

Some of the main aspects of the present invention are summarized herein. Additional aspects are described in the Detailed Description of the Invention, Examples, Drawings and Claims sections of this disclosure. The description of each section of this disclosure is intended to be read in conjunction with the other sections. Furthermore, the various embodiments described in each section of the disclosure can be combined in different ways, and all such combinations are intended to fall within the scope of the present invention.

FIG. 1. The spinal cord of EAE mice contains two subsets of neutrophils distinguishable by ICAM1 surface expression. a, Flow cytometry gating strategy used to analyze neutrophils (CD45⁺CD11b⁺Ly6G⁺CD19⁻CD3ε⁻) from the spinal cord or blood of mice euthanized 15 days after immunization either with or without MOG₃₅₋₅₅ (EAE or sham, respectively). At this time point, the EAE mice had clinical scores ranging from 0.5 to 3 (mean, 1.9±0.2). Naïve mice were used as additional controls. Dead cells and doublets were excluded. b, Quantification of the data in a revealing an increase of neutrophils in the spinal cord and blood of EAE and sham-treated mice. For the spinal cords, counts were normalized to CD45⁻ cells as an internal control. Stars indicate significant increases from both the naïve and sham-treated mice ( [symbol] ) or from the naïve mice only ( [symbol] ), as determined by ANOVA and post hoc Student's t test (P≤0.0089). Sample size for spinal cord: 21 (EAE), 10 (sham, naïve). Sample size for blood: 13 (EAE), 7 (sham), 6 (naïve). c, Flow cytometric analysis of ICAM1 on neutrophils from the spinal cord or blood of EAE and control mice. Data were gated as in a. d, Quantification of the data in c revealing a strong increase of ICAM1 expression on neutrophils isolated from the spinal cord of EAE mice. Left charts, counts of ICAM1⁺ and ICAM1⁻ neutrophils in the spinal cord and blood. Right charts, median fluorescence intensity (MFI) obtained for ICAM1 when gated on the whole population of neutrophils or only on those positive for ICAM1. Stars indicate significant increases of ICAM1⁺ neutrophils from both the naïve and sham-treated mice ( [symbol] ) or from the naïve mice only ( [symbol] ), as determined by ANOVA and post hoc Student's t test (P≤0.0009). Sample size as in b. e, Flow cytometric quantification of CD11b and CD45 on ICAM1⁺ and ICAM1⁻ neutrophils from the spinal cord of EAE mice. Stars indicate significant increases according to Student's t test (P<0.0001). The right chart shows a positive correlation between CD11b and CD45 expression, as calculated with the Pearson correlation test. Sample size: 15 per group. f, Confocal images showing different nuclear morphologies in spinal cord neutrophils isolated by FACS and stained with DAPI. Scale bar: 1 μm. g, Frequency of the different nuclear morphologies in ICAM1⁺ and ICAM1⁻ neutrophils separately purified from the spinal cord of EAE mice by FACS. No intergroup difference was observed (Student's t test, P>0.1). Sample size: 50-160 nuclei were counted per cell subset and per mouse (total of 5 mice).

FIG. 2. ICAM1⁺ and ICAM1⁻ neutrophils are differently distributed in the spinal cord during EAE. a, Confocal images of a spinal cord section from a Catchup×Ai6 mouse with EAE (day 15) showing ZsGreen⁺ neutrophils (arrows) infiltrated in meningeal and submeningeal inflammatory foci. Note the multilobed morphology of their nucleus stained with DAPI. The right images are higher magnification views of the box in the left image. The dashed line delineates the leptomeninges. Scale bar: left, 100 μm; right, 10 μm. b, High magnification of a ZsGreen⁺ neutrophil immunostained for Ly6G. Scale bar: 5 μm. c, An ICAM1⁻ neutrophil with a rod-shaped morphology crawling on the luminal surface of an ICAM1⁺ capillary (white, immunostaining for laminin revealing the endothelial basal membrane). Right images are y-z sections taken at the dashed line. Scale bar: 5 μm. d, Extravasated ICAM1⁺ neutrophils with an amoeboid morphology. Scale bar: 5 μm.

FIG. 3. ICAM1⁺ neutrophils have a distinct transcriptional profile suggestive of a capacity for immunomodulation and antigen presentation. a, Heat map of mRNAs differentially expressed in ICAM1⁺ and ICAM1⁻ neutrophils (N⁺, N⁻) compared to each other or to CD11b⁺CD11c⁻Ly6G⁻ macrophages (Mc) and CD11b⁺CD11c⁺Ly6G⁻ dendritic cells (DC). These cells were simultaneously purified from the spinal cord of EAE mice by FACS and analyzed by DNA microarray. The hierarchical clustering dendrogram shows the degree of similarity among the samples (biological duplicates). The grey scale indicates the hybridization signal intensity. The criteria used for comparison were as follows: fold change 3; hybridization signal >200; Student's t test, P≤0.05. b, Frequency distribution, according to biological function, of the 343 mRNAs identified as enriched in ICAM1⁺ neutrophils. c, Fold difference in the hybridization signals for mRNAs of three selected categories, as compared between ICAM1⁺ and ICAM1⁻ neutrophils. d, Fold difference in the hybridization signals for neutrophil-specific mRNAs, as compared between neutrophils (ICAM1⁺ and ICAM1⁻) and macrophages. e, Signaling proteins expressed or upregulated in ICAM1⁺ neutrophils, supporting the concept that these cells acquire immunostimulatory capacities. f, Schematic of the MHCII pathway showing that all of the proteins are upregulated in ICAM1⁺ neutrophils, suggesting the acquisition of antigen-presenting capacity. Abbreviations: ER, endoplasmic reticulum; MIIC, MHCII compartment.

FIG. 4. A fraction of ICAM1⁺ neutrophils exhibit surface proteins involved in antigen presentation. a, Representative cytometry plots showing the gates used to analyze MHCII and co-stimulatory molecules on neutrophils (CD11b⁺Ly6G⁺CD11c⁻CD19⁻CD3ε⁻) from the spinal cord of EAE mice. b, Quantification of the results in a revealing increases of MHCII and co-stimulatory molecules on subpopulations of ICAM1⁺ neutrophils. Stars indicate significant differences from the corresponding ICAM1⁻ population ( [symbol] ) or from all the other populations ( [symbol] ), as determined by the Kruskal-Wallis test and post hoc Wilcoxon test (P≤0.0051). Sample size: 6 mice.

FIG. 5. ICAM1⁺MHCII⁺ neutrophils express less Ly6G, but do not differentiate into Ly6G⁻ monocytic cells. a-c, Quantification of Ly6G expression in neutrophil subsets isolated from the spinal cord of EAE mice at day 14 by microarray, RT-qPCR or flow cytometry, respectively. Note the reduction of Ly6G expression, which was most pronounced in the MHCII^{hi} fraction. Stars indicate significant decreases from the other group(s) according to Student's t or Wilcoxon test (a, P<0.0001; b, P=0.046; c, P<0.032). Sample size: 2 (a), 4 (b) or 6 (c) per group. d, Flow cytometric analysis revealing the nature of ZsGreen⁺ cells in the spinal cord of Catchup×Ai6 mice with EAE. ZsGreen expression was analyzed in neutrophils (CD11b⁺Ly6G⁺), dendritic cells (CD45^{hi}CD11b⁺CD11c⁺), macrophages (CD45^{hi}CD11b⁺CD11c⁻), microglia (CD45^{lo}CD11b⁺), B cells (CD19⁺) and T cells (CD3ε⁺). e, Deconvolved confocal images of an Iba1⁺ phagocyte (red) containing engulfed ZsGreen⁺ particles. The right image is a 3D reconstruction of the left image with a sliced portion (dashed lines) showing that the ZsGreen⁺ particles are inside the cytoplasm. Scale bar: 1 μm.

FIG. 6. Neutrophils form immune synapses with lymphocytes in the spinal cord of EAE mice. a, Low-magnification confocal image of the spinal cord and meninges in a EAE mouse at day 16 post-immunization. Neutrophils (ZsGreen⁺) and T cells (CD3ε⁺) infiltrated the subpial parenchyma, while B cells (B220⁺) remained in the meninges (under the dashed line). Scale bar: 10 μm. b, Close-up images of neutrophils making synapses (arrows) with T or B cells (top and bottom panels, respectively). Scale bar: 2 μm. c, Frequency of immune synapses in the spinal cord parenchyma and meninges. Data are expressed as the percentage of a given synapse (y axis) among a given leukocyte population (x axis). Sample size: 359-2167 cells were counted per region per animal (total of 4 EAE mice). d, Super-resolution micrographs of a neutrophil-T cell synapse (only 3 optical sections are shown). ICAM1 and CD3ε were acquired in STED mode, whereas ZsGreen and DAPI were acquired in confocal mode. Scale bar: 2 μm. e, Three-dimensional rendering of the cells in d (all optical sections are shown). Note the absence of ICAM1 at the point of synapse (dashed line). Scale bar: 2 μm.

FIG. 7. ASPRV1 is a neutrophil-specic gene expressed in EAE. a, Quantification of ASPRV1 mRNA by RT-qPCR in leukocytes isolated by FACS from the spinal cord of EAE mice at day 15 post-induction. CD3ε⁺ T cells (T) were from either mice immunized with MOG₃₅₋₅₅ (active EAE), mice transplanted with encephalitogenic T cells (passive EAE), or 2D2 mice that developed EAE after PTX injection. CD19⁺ B cells (B), CD45^{lo}CD11b⁺ microglia (Mic), CD45^{hi}CD11b⁺CD11c⁺ dendritic cells (DC), CD45^{hi}CD11b⁺ macrophages (Mc) and Ly6G⁺ neutrophils expressing or not ICAM1 (N⁺ and N⁻, respectively) were from mice with active EAE. Sample size: 4 per group. b, ASPRV1 protein (˜32 kDa) detected by Western blotting in Percoll-enriched neutrophils from the bone marrow of wild-type mice, but not of ASPRV1^{-/-} mice, nor in the mononuclear cell fraction from either genotype. Total loading per well: 20 μg protein. Actin (42 kDa) was used as a control for protein loading. c, Quantification of ASPRV1 mRNA by RT-qPCR in whole spinal cord from mice with different forms of EAE or from controls, i.e. mice treated with PBS, PTX or CFA, and 2D2 mice that did not develop EAE after PTX injection (2D2 without [w/o] EAE). Stars indicate significant differences from PBS, PTX and CFA (for active and passive EAE) or from 2D2 without EAE (for 2D2 mice) at the same time point (Wilcoxon test, P<0.018). Sample size: 6 (PBS), 7-13 (PTX), 3-8 (CFA), 8 (active EAE), 8 (passive EAE), 6 (2D2 with EAE), 5 (2D2 without EAE). d, Spearman analysis showing a strong positive correlation between ASPRV1 and Ly6G mRNA expression in the spinal cord during active EAE. Sample size: 32. e, RT-qPCR analysis of ASPRV1 mRNA in freshly isolated human blood cells. Star indicates a significant difference from the other groups (Wilcoxon test, P=0.0005). Sample size: 2-10 per group. f, RT-qPCR analysis of ASPRV1 mRNA in post-mortem brain samples from control individuals (normal) or patients with typical or severe forms of MS. NAWM: normal-appearing white matter. Star indicates a significant difference from the other groups (Wilcoxon test, P=0.006). Sample size: 13-16 per group.

FIG. 8. ASPRV1 is required for the chronic phase of EAE induced with a B cell-dependent myelin antigen (bMOG), but not for initial neutrophil recruitment or T cell priming. a, Kaplan-Meier plot of EAE incidence in wild-type (ASPRV1^{-/+}) and ASPRV1-deficient mice (ASPRV1^{-/-}) after immunization with either MOG₃₅₋₅₅ or bMOG. Shaded area represents the 95% pointwise confidence interval. For sample size and statistical testing, see incidence in c. b, Severity of EAE over time by group versus time from appearance of first symptoms. P-value shown on graph was calculated by two-way ANOVA with repeated measures using rank-transformed scores. Stars indicate differences in post-hoc testing by time point (Wilcoxon test, P<0.0386). Only mice that had developed EAE are included (see incidence in c for sample size). c, Statistical data extracted from the clinical score of knockout and wild-type mice. Means were compared by Fisher's exact test (for recovery), by the log-rank test (for incidence and mortality), or otherwise by Wilcoxon test. d, Flow cytometric counts of immune cells in the spinal cord of ASPRV1^{-/-} and wild-type mice before immunization with bMOG (naïve) or after at day 8 (EAE pre-onset), 13 (EAE peak) or 21 (EAE chronic phase). Counts were normalized to CD45⁻ cells as an internal control. Stars indicate significant differences from ASPRV1^{+/+} mice (Wilcoxon test, P<0.0454). Abbreviations: Mic, CD45^{lo}CD11b⁺ microglia; N, Ly6G⁺ neutrophils; Mc, CD45^{hi}CD11b⁺CD11c macrophages; DC, CD45^{hi}CD11b⁺CD11c⁺ dendritic cells; T, CD3ε⁺ T cells; B, CD19⁺ B cells. Sample size per group: 4-6 (naïve), 7 (pre-onset), 9-22 (peak), 7-10 (chronic). e, Counts of the ICAM1⁻ (intravascular) and ICAM1⁺ (extravascular) subpopulations of macrophages and neutrophils showing no intergroup difference, except for ICAM1⁺ macrophages at the peak of bMOG EAE. Star indicates a significant difference from ASPRV1^{+/+} mice (Wilcoxon test, P=0.0374). Sample size as in d.f, Counts of blood neutrophils showing that bMOG immunization induces a similar mobilization of neutrophils between the genotypes. Sample size per group: 3-5 (naïve), 7 (pre-onset), 7-10 (peak). g, Counts of Tₕ17 (IL-17⁺) and Tₕ1(IFN-γ+) cells in inguinal lymph nodes (LN) at day 9 post-immunization with bMOG suggesting that there were no intergroup difference in T cell priming. Intracellular staining was performed on freshly collected LN cells after a 4-h stimulation with phorbol myristate acetate and ionomycin in the presence of brefeldin A. Sample size: 14-17 per group.

FIG. 9. List of antibodies used for flow cytometry

FIG. 10. List of antibodies used for immunohistochemistry.

FIG. 11. Cre-mediated excision of H2-Ab1 gene in neutrophils does not reduce H2-Ab1 mRNA and surface MHCII. A, Quantification of the H2-Ab1 gene exon I (floxed) by qPCR in neutrophil subsets purified by FACS from the spinal cord of Ly6G^{cre/cre}(Catchup) and Ly6G^{cre/cre}H2-Ab^{fl/fl} mice with EAE. Data were normalized to exon 3 (not floxed). Stars indicate significant excision (>80%), as determined by Wilcoxon test (P<0.0001). Sample size: 10-12 mice per group. B, Quantification of H2-Ab1 mRNA by RT-qPCR in spinal cord neutrophils using primers directed at exon 1. Data were normalized to HPRT1 mRNA and revealed no intergenotype difference. Sample size: 5-6 mice per group. C, D, Flow cytometric analysis of MHCII showing no difference in the percentage of neutrophils that were positive for MHCII (c) nor in the amount of MHCII on the surface of these cells (d). MFI, median fluorescence intensity. Sample size: 5-6 mice per group.

FIG. 12. B cells are essential for EAE induction with bMOG. A, EAE score over time in B1-8^{+/+}Jκ^{-/-} mice and wild-type controls (WT) immunized with bMOG. Star indicates a significant intergenotype difference per time point identified by post hoc Wilcoxon test (P<0.0001). Sample size: 6 (WT), 4 (B1-8⁺/Jκ^{-/-}). B, Detection of MOG₁₋₁₂₅-specific plasma cells by ELISpot in bone marrow (BM) from WT and B1-8^{+/+}*Jκ^{-/-} mice at day 29 post-immunization. Star indicates a significant difference, as determined by Student's t test (P<0.0001). Sample size: 6 (WT), 3 (B1-8^{+/+}Jκ^{-/-}). Each mouse was tested in triplicate.

FIG. 13. Neutrophils synthesize and secrete a short form of ASPRV1 (˜28 kDa). A, ASPRV1 was detected (arrows) by Western blotting in both cell extracts and culture supernatants of neutrophils purified from the bone marrow of wild-type mice (Asprv1^{+/+}), but not of ASPRV1-deficient mice (Asprv1^{-/-}). Cells were unstimulated (PBS) or stimulated for 30 min with 100 μM phorbol myristate acetate (PMA). Samples from 2×106 neutrophils were loaded per well. Non-specific bands (NB) served as loading controls. B, Quantification of the Western blots for ASPRV1 (shown in A) by optical densitometry showed that PMA increased the secretion of ASPRV1. *Significantly different from PBS (Wilcoxon test, p=0.04; n=3 per group).

### DETAILED DESCRIPTION OF INVENTION

### I—Definitions

The headings provided herein are not limitations of the various aspects of the disclosure, which can be added by reference to the specification as a whole. Accordingly, the terms defined immediately below are more fully defined by reference to the specification in its entirety.

As used herein the term “ASPRV1” refers to the ASPRV1 protein or fragment thereof, nucleic acids encoding the ASPRV1 protein or a fragment of such ASPRV1 nucleic acid or complement thereof.

As used herein the term “ASPRV1 protein” encompasses human ASPRV1 protein, mouse ASPRV1, primate ASPRV1, bovine ASPRV and any other species in which ASPRV1 has been identified. The term “ASPRV1 protein” also encompasses the full protein, the protein resulting from auto-cleavage and active fragments of the protein. The term “ASPRV1 protein” also relates to an ASPRV1 dimer. In a preferred embodiment, the term “ASPRV1 protein” refers to human ASPRV1.

As used herein, the term “ASPRV1 fragment” includes an active fragment as well as a short form of ASPRV1. The term “active fragment” with reference to a protein relates to a portion of a protein that retains an activity of the protein. With respect to the ASPRV1 protein, an active fragment may include a portion of the protein that retains the aspartic peptidase activity. For example, for the human ASPRV1 protein (SEQ ID NO.:4) or mouse ASPRV1 protein (SEQ ID NO.:2), an active fragment encompasses the catalytic domain. In the case of human ASPRV1, amino acids 191-326 possesses autoprocessing activity and is catalytically active (Bernard et al., J Invest Dermatol 125:278-287, 2005). In the case of mouse ASPRV1, amino acids 189 to 324 possesses autoprocessing activity and is catalytically active (Matsui et al., J. Biol Chem. 281, 27512-27515, 2006). The term “short form of ASPRV1” includes for example, SEQ ID NO.:5 and SEQ ID NO.:6.

The term “biological sample” as used herein is a sample obtained from the mammal and encompasses, for example and without limitations, blood (including whole sample, plasma and serum), cerebrospinal fluid as well as various tissues (e.g., post-mortem CNS samples, etc.).

Wherever aspects are described herein with the language “comprising,” otherwise analogous aspects described in terms of “consisting of” and/or “consisting essentially of” are also provided.

The terms “polynucleotide,” “oligonucleotide,” “nucleic acid,” “nucleic acid molecule,” and “gene” are used interchangeably herein to refer to polymers of nucleotides of any length, and ribonucleotides, deoxyribonucleotides, analogs thereof, or mixtures thereof.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

A polypeptide, antibody, polynucleotide, vector, cell, or composition which is “isolated” is a polypeptide, polynucleotide, or composition which is in a form not found in nature. Isolated polypeptides, polynucleotides, or compositions include those that have been purified to a degree that they are no longer in a form in which they are found in nature. In some aspects, a polypeptide, polynucleotide, or composition that is isolated is substantially pure.

The term “percent sequence identity” between two polypeptide or polynucleotide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm.

BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that align with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment, e.g., from the EBI. It will also be appreciated that the final alignment used to calculate percent sequence identity can be curated either automatically or manually.

As used herein, the term “fragment” with reference to a protein relates to portion of at least 6 consecutive amino acids of the given polypeptide including, e.g., a peptide of at least 8, 9, 10, 11, 12, 13, 14, 15, 16, 17, 18, 19 and 20 consecutive amino acids of the polypeptide. A fragment of the human ASPRV1 protein (SEQ ID NO.:4), will encompass fragments of between 6 to 342 consecutive amino acids of SEQ ID NO.:4. The term “fragment” encompasses “active fragments”. A fragment of the mouse ASPRV1 protein (SEQ ID NO.:2), will encompass fragments of between 6 to 338 consecutive amino acids of SEQ ID NO.:4. The fragment may be immunogenic and may be used for the purpose, for example, of generating an immune response.

As used herein, the term “fragment” with reference to a nucleic acid relates to a sequence of at least 10 consecutive nucleotides of such nucleic acid and encompasses a sequence of at least 15 nucleotides and a sequence of at least 20 nucleotides. The nucleic acid fragment of the present invention may be used, for example, as a probe or primer.

The term “antibody” refers to intact antibody, monoclonal or polyclonal antibodies. The term “antibody” also encompasses multispecific antibodies such as bispecific antibodies. Human antibodies are usually made of two light chains and two heavy chains each comprising variable regions and constant regions. The light chain variable region comprises 3 CDRs, identified herein as CDRL1 or L1, CDRL2 or L2 and CDRL3 or L3 flanked by framework regions. The heavy chain variable region comprises 3 CDRs, identified herein as CDRH1 or H1, CDRH2 or H2 and CDRH3 or H3 flanked by framework regions. The CDRs of the humanized antibodies of the present invention have been identified using the Kabat and Chotia definitions (e.g., CDRH2 set forth in SEQ ID NO.:56). However, others (Abhinandan and Martin, 2008) have used modified approaches based loosely on Kabat and Chotia resulting in the delineation of shorter CDRs (e.g., CDRH2 set forth in SEQ ID NO.:6).

The term “antigen-binding fragment”, as used herein, refers to one or more fragments of an antibody that retain the ability to bind to an antigen. It has been shown that the antigen-binding function of an antibody can be performed by fragments of an intact antibody. Examples of binding fragments encompassed within the term “antigen-binding fragment” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR), e.g., V_{H} CDR3. Furthermore, although the two domains of the Fv fragment, V_{L} and V_{H}, are coded for by separate genes, they can be joined, using recombinant methods, by a synthetic linker that enables them to be made as a single polypeptide chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Furthermore, the antigen-binding fragments include binding-domain immunoglobulin fusion proteins comprising (i) a binding domain polypeptide (such as a heavy chain variable region, a light chain variable region, or a heavy chain variable region fused to a light chain variable region via a linker peptide) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The hinge region may be modified by replacing one or more cysteine residues with serine residues so as to prevent dimerization. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

The term “humanized antibody” encompasses fully humanized antibody (i.e., frameworks are 100% humanized) and partially humanized antibody (e.g., at least one variable domain contains one or more amino acids from a human antibody, while other amino acids are amino acids of a non-human parent antibody). Typically a “humanized antibody” contains CDRs of a non-human parent antibody (e.g., mouse, rat, rabbit, non-human primate, etc.) and frameworks that are identical to those of a natural human antibody or of a human antibody consensus. In such instance, those “humanized antibodies” are characterized as fully humanized. A “humanized antibody” may also contain one or more amino acid substitutions that have no correspondence to those of the human antibody or human antibody consensus. Such substitutions include, for example, back-mutations (e.g., re-introduction of non-human amino acids) that may preserve the antibody characteristics (e.g., affinity, specificity etc.). Such substitutions are usually in the framework region. A “humanized antibody” optionally also comprise at least a portion of a constant region (Fc) which is typically that of a human antibody. Typically, the constant region of a “humanized antibody” is identical to that of a human antibody. However, some modifications may be done in the Fc portion order to conjugate a specific drug or to modify its affinity or therapeutic activity.

The term “chimeric antibody” refers to an antibody having non-human variable region(s) and human constant region.

The term “hybrid antibody” refers to an antibody comprising one of its heavy or light chain variable region (its heavy or light chain) from a certain type of antibody (e.g., humanized), while the other of the heavy or light chain variable region (the heavy or light chain) is from another type (e.g., murine, chimeric).

In some instances, the antibody or antigen-binding fragment of the present invention may be conjugated with cargo moiety such as a detectable moiety (i.e., for detection or diagnostic purposes) or a therapeutic moiety (for therapeutic purposes).

More particularly, the antibody or antigen-binding fragment thereof may be conjugated with a therapeutic moiety such as a drug, cytotoxic moiety, cytostatic agent or chemotherapeutics, and may encompass, without limitation, Yttrium-90, Scandium-47, Rhenium-186, lodine-131, Iodine-125, and many others recognized by those skilled in the art (e.g., lutetium [Lu], bismuth [Bi²³], copper [Cu⁶⁷], 5-fluorouracil, adriamycin, irinotecan, taxanes, Pseudomonas endotoxin, ricin, auristatins [e.g., monomethyl auristatin E, monomethyl auristatin F], maytansinoids [e.g., mertansine, maytansine, emtansine], amanitin, chalicheamicin, ravtansine, pyrrolobenzodiazepine and other toxins).

Alternatively, in order to carry out the methods of the present invention and as known in the art, the antibody or antigen-binding fragment of the present invention (conjugated or not) may be used in combination with a second molecule (e.g., a secondary antibody, etc.) that is able to specifically bind to the antibody or antigen-binding fragment of the present invention and which may carry a desirable detectable, diagnostic or therapeutic moiety.

A “detectable moiety” is a moiety detectable by spectroscopic, photochemical, biochemical, immunochemical, enzymatic, chemical and/or other physical means. A detectable moiety may be coupled either directly and/or indirectly (for example via a linkage, such as, without limitation, a DOTA or NHS linkage) to antibodies and antigen-binding fragments thereof of the present invention using methods well known in the art. A wide variety of detectable moieties may be used, with the choice depending on the sensitivity required, ease of conjugation, stability requirements and available instrumentation. A suitable detectable moiety includes, but is not limited to, a fluorescent label, a radioactive label (e.g., without limitation, ¹²⁵I, In¹¹¹, Tc⁹⁹, ¹¹³I and including positron emitting isotopes for PET scanner), a nuclear magnetic resonance active label, a luminiscent label, a chemiluminescent label, a chromophore label, an enzyme label (e.g., without limitation, horseradish peroxidase, alkaline phosphatase), quantum dots and/or a nanoparticle. Detectable moiety may cause and/or produce a detectable signal thereby allowing for a signal from the detectable moiety to be detected. In some instances, a “detectable moiety” may also have some therapeutic effect.

In another aspect, the present invention provides a cell that may comprise and/or may express the antibody described herein. The cell may comprise a nucleic acid encoding a light chain variable region and a nucleic acid encoding a heavy chain variable region and may be capable of expressing, assembling and/or secreting an antibody or antigen-binding fragment thereof.

The antibodies that are disclosed herein can be obtained by a variety of methods familiar to those skilled in the art, such as by hybridoma methodology, from transgenic animals engineered to expressed antibodies such as human antibodies, by screening antibody or antigen-binding fragment libraries etc.

In an exemplary embodiment of the invention, the antibodies may be produced by the conventional hybridoma technology, where a mouse is immunized with an antigen, spleen cells isolated and fused with myeloma cells lacking HGPRT expression and hybrid cells selected by hypoxanthine, aminopterin and thymine (HAT) containing media. Once the sequence of an antibody or an antigen-binding fragment is known, the antibody may be produced by recombinant DNA methods in cell lines. Different host cells that have specific cellular machinery and characteristic mechanisms for post-translational activities (e.g., CHO, HeLa, MDCK, HEK293, and W138) are available commercially and from the American Type Culture Collection (ATCC) and may be chosen to ensure the correct modification and processing of an antibody or antigen-binding fragment thereof.

In order to express the antibodies, nucleotide sequences able to encode any one of a light and heavy immunoglobulin chains described herein or any other may be inserted into an expression vector, i.e., a vector that contains the elements for transcriptional and translational control of the inserted coding sequence in a particular host. These elements may include regulatory sequences, such as enhancers, constitutive and inducible promoters, and 5′ and 3′ un-translated regions. Methods that are well known to those skilled in the art may be used to construct such expression vectors. These methods include in vitro recombinant DNA techniques, synthetic techniques, and in vivo genetic recombination.

A variety of expression vector/host cell systems known to those of skill in the art may be utilized to express a polypeptide or RNA derived from nucleotide sequences able to encode any one of a light and heavy immunoglobulin chains described herein. These include, but are not limited to, microorganisms such as bacteria transformed with recombinant bacteriophage, plasmid, or cosmid DNA expression vectors; yeast transformed with yeast expression vectors; insect cell systems infected with baculovirus vectors; plant cell systems transformed with viral or bacterial expression vectors; or animal cell systems. For long-term production of recombinant proteins in mammalian systems, stable expression in cell lines may be effected. For example, nucleotide sequences able to encode any one of a light and heavy immunoglobulin chains described herein may be transformed into cell lines using expression vectors that may contain viral origins of replication and/or endogenous expression elements and a selectable or visible marker gene on the same or on a separate vector. The invention is not to be limited by the vector or host cell employed. In certain embodiments of the present invention, the nucleotide sequences able to encode any one of a light and heavy immunoglobulin chains described herein may each be ligated into a separate expression vector and each chain expressed separately. In another embodiment, both the light and heavy chains able to encode anyone of alight and heavy immunoglobulin chains described herein may be ligated into a single expression vector and expressed simultaneously.

Alternatively, RNA and/or polypeptide may be expressed from a vector comprising nucleotide sequences able to encode any one of a light and heavy immunoglobulin chains described herein using an in vitro transcription system or a coupled in vitro transcription/translation system respectively.

In general, host cells that contain nucleotide sequences able to encode any one of a light and heavy immunoglobulin chains described herein and/or that express a polypeptide encoded by the nucleotide sequences able to encode any one of a light and heavy immunoglobulin chains described herein, or a portion thereof, may be identified by a variety of procedures known to those of skill in the art. These procedures include, but are not limited to, DNA/DNA or DNA/RNA hybridizations, PCR amplification, and protein bioassay or immunoassay techniques that include membrane, solution, or chip based technologies for the detection and/or quantification of nucleic acid or amino acid sequences. Immunological methods for detecting and measuring the expression of polypeptides using either specific polyclonal or monoclonal antibodies are known in the art. Examples of such techniques include enzyme-linked immunosorbent assays (ELISAs), radioimmunoassays (RIAs), and fluorescence activated cell sorting (FACS). Those of skill in the art may readily adapt these methodologies to the present invention.

Host cells comprising nucleotide sequences able to encode any one of a light and heavy immunoglobulin chains described herein may thus be cultured under conditions for the transcription of the corresponding RNA (mRNA, etc.) and/or the expression of the polypeptide from cell culture. The polypeptide produced by a cell may be secreted or retained intracellularly depending on the sequence and/or the vector used. In an exemplary embodiment, expression vectors containing nucleotide sequences able to encode any one of a light and heavy immunoglobulin chains described herein may be designed to contain signal sequences that direct secretion of the polypeptide through a prokaryotic or eukaryotic cell membrane.

As used herein, “pharmaceutical composition” means therapeutically effective amounts of the agent together with pharmaceutically acceptable carrier, diluents, preservatives, solubilizers, emulsifiers, adjuvant and/or carriers. A “therapeutically effective amount” as used herein refers to that amount that provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” or “pharmaceutical carrier” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the EDso (the dose therapeutically effective in 50% of the population) and LDso (the dose lethal to 50% of the population) statistics.

Any of the therapeutic compositions described above may be applied to any subject in need of such therapy, including, but not limited to, mammals such as dogs, cats, cows, horses, rabbits, monkeys, and humans.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

The term “treatment” for purposes of this disclosure refers to both therapeutic treatment and prophylactic or preventative measures. The term “treatment” encompasses ameliorating the symptoms associated with the disease or condition. Those in need of treatment include those already with the disorder as well as those prone to have the disorder or those in whom the disorder is to be prevented. Particularly, mammals in need include those with health conditions involving neutrophils such as neuroinflammation, autoimmune demyelination and other inflammatory and autoimmune diseases.

### II—Aspects and Exemplary Embodiments of the Invention

The Applicant has identified aspartic peptidase retroviral-like 1 (ASPRV1) as a neutrophil-specific marker.

ASPRV1 and reagents that specifically binds ASPRV1 may thus serve for the identification of neutrophils or assessment of their activity. Since the ASPRV1 protein and mRNA were observed in the neutrophils of mouse and human, it is possible that this protein be also expressed in neutrophils of other mammals such as primates (non-human primates), bovines, rats, cats and dogs. The invention may therefore be applicable to species other than human and mouse.

The present invention therefore relates in one aspect thereof to a method for identifying neutrophils in a biological sample obtained from a mammal. The method may comprise detecting ASPRV1 protein or a fragment thereof. Alternatively, the method may comprise detecting an ASPRV1-encoding nucleic acid or a fragment or complement thereof. Nucleic acids may be detected with specific probes or with primers adapted for amplification of the desired ASPRV1 nucleic acid sequence. More particularly, ASPRV1 nucleic acids may be detected by the formation of a complex between the ASPRV1 nucleic acid and probes. The method of the present invention encompasses detecting mammalian neutrophils, such as neutrophils from rodents, humans, or primates.

In accordance with the present invention, the ASPRV1 protein or fragment thereof may be detected with an antibody or an antigen-binding fragment thereof that is capable of specific binding. More particularly, ASPRV1 may be detected by the formation of a complex between the protein and the antibody or antigen-binding fragment thereof.

It may be advantageous for an antibody or an antigen-binding fragment thereof to be capable of binding the ASPRV1 protein or fragment of different species in order to facilitate drug development or their use in different types of assays.

As such, the method of the present invention encompasses anti-ASPRV1 antibodies or antigen-binding fragments thereof having dual specificity including those that are capable of specific binding to ASPRV1 from one or multiple species such as human, primates and rodents, to mouse a rodent ASPRV1 (e.g., mouse ASPRV1), to a primate ASPRV1 or to two or more of human ASPRV1, mouse ASPRV1 and primate ASPRV1.

As the ASPRV1 protein is an aspartic peptidase, it may also be possible to detect its presence by assessing its enzymatic activity. For example, the ASPRV1 protein expressed in stratified epithelia cleaves profilaggrin. It is therefore possible that the ASPRV1 protein expressed in neutrophils be also capable of processing profilaggrin and other substrats even if they may not be relevant for the neutrophil activity or phenotype.

The ASPRV1 protein or fragment thereof may also be detected by peptide sequencing or other methods known to a person skilled in the art.

Nucleic acids encoding ASPRV1 (mRNA, cDNA, etc.) or nucleic acid complements (non-coding strand) or fragments may be detected by methods known to a person skilled in the art which may involve, for example, amplification by polymerase chain reaction and/or hybridization with probes.

The Applicant also found that ASPRV1 is specifically expressed by neutrophils and correlates with neutrophil infiltration in demyelinating autoimmune diseases.

Therefore, the present invention relates in an additional aspect to a method for the diagnosis or prognosis of a demyelinating disease in a mammal. The method may comprise detecting ASPRV1 or detecting neutrophils expressing ASPRV1. The detection may be performed by assessing the presence of ASPRV1 in a sample obtained from the mammal. Such a sample may originate, for example, from blood or tissues of the mammal. The invention may be extended to diseases caused or related to neutrophils such as inflammatory diseases and other autoimmune diseases.

It would be advantageous to target neutrophils with a compound that specifically binds to the ASPRV1 protein or nucleic acids expressing ASPRV1 in order to detect this specific cell type or deliver a drug to inhibit their function in vitro, ex vivo or in vivo.

As such, the present invention relates in an additional aspect to a method of targeting neutrophils in a mammal. The method may comprise administering a compound that is capable of specific binding to the ASPRV1 protein or to a fragment thereof or a compound that is capable of specific binding to a nucleic acid encoding the ASPRV1 protein, a nucleic acid complement or a nucleic acid fragment thereof. The method may further comprise administering a compound that is capable of inhibiting ASPRV1 enzymatic activity.

Such compounds may include for example, a peptide, a peptidomimetic, a small molecule and more particularly, an antibody or an antigen-binding fragment thereof that specifically binds to the ASPRV1 protein or to a fragment thereof. The anti-ASPRV1 antibody or antigen-binding fragment may, for example, inhibit the dimerization of the ASPRV1 protein or of an ASPRV1 fragment, may inhibit the enzymatic activity of the ASPRV1 protein or of the fragment thereof, may inhibit auto-cleavage of the ASPRV1 protein or may block the interaction of ASPRV1 with a ligand.

Such compounds may also include, for example, those that bind to a nucleic acid encoding ASPRV1 and inhibit its expression, trigger its degradation or result in an inhibition of the gene. Exemplary embodiments of such compounds may include siRNAs, antisenses and the like.

The present invention also relates to a method for treating a disease associated with neutrophils in a mammal in need thereof. The method may comprise detecting ASPRV1 in a biological sample obtained from the mammal having or suspected of having a disease (e.g. a disease associated with neutrophils). The level of ASPRV1 detected in the biological sample may be compared to a biological sample obtained from the same individual prior to or after the onset of symptoms. The level of ASPRV1 may also be compared with the normal level observed in the general population.

An increase in the level of ASPRV1 may indicate a disease associated with neutrophils. An increased level of ASPRV1 may indicate a worsening in the severity of the disease. The method may therefore comprise administering a drug that modulates the activity of neutrophils or that reduces their numbers.

The present invention provides in a further aspect thereof, an antibody or an antigen-binding fragment that is capable of specific binding to the ASPRV1 protein or to a fragment thereof. Such antibody may be used for detection purposes, diagnostic or prognostic purposes or for treatment of neutrophil-associated diseases or disorders. Nucleic acids and vectors encoding the 3 light chain CDRs, the 3 heavy chain CDRs, the light chain variable region, the heavy chain variable region or the complete antibody sequence are also encompassed by the present invention.

The antibodies or antigen-binding fragments thereof may comprise monoclonal antibodies, polyclonal antibodies, chimeric antibodies, humanized antibodies, human antibodies as well as antigen-binding fragments. Antibodies or antigen-binding fragments encompassing permutations of the light and/or heavy chains between a monoclonal, chimeric, humanized or human antibody are also encompassed herewith.

The antibodies or antigen-binding fragments of the present invention may thus comprise amino acids of a human constant region and/or framework amino acids of a human antibody.

Antibodies and/or antigen-binding fragments of the present invention may originate, for example, from a mouse, rat or any other mammal or from other sources such as through recombinant DNA technologies.

Pharmaceutical compositions are also encompassed by the present invention. The pharmaceutical composition may comprise a compound that is capable of specific binding to the ASPRV1 protein or to a fragment thereof, a compound that is capable of specific binding to a nucleic acid encoding the ASPRV1 protein, a nucleic acid complement or a nucleic acid fragment thereof or a compound that is capable of inhibiting ASPRV1 enzymatic activity and a pharmaceutically acceptable carrier.

Pharmaceutical compositions comprising an anti-ASPRV1 antibody or an antigen-binding fragment and a pharmaceutically acceptable carrier are particularly contemplated.

Other aspects of the invention relate to a composition that may comprise the antibody or antigen-binding fragment described herein and a carrier.

The treatment of mammals having a disease associated with neutrophils using a compound that targets ASPRV1 is also contemplated. The present invention therefore provides in an additional aspect a method of treating a mammal having an autoimmune disorder associated with neutrophils. The method may comprise administering a compound that inhibits the ASPRV1 protein, an ASPRV1 active fragment or a nucleic acid encoding the ASPRV1 protein. The method may also comprise administering a compound that sequester a secreted form of ASPRV1.

In accordance with the present invention, compounds that may be useful for treatment includes, for example, a compound that binds to ASPRV1. Such compound may also include those that inhibit the enzymatic activity of ASPRV1, inhibit dimerization of ASPRV1, or those comprising a nucleic acid portion that inhibits the expression of ASPRV1 or induces the degradation or silencing of the nucleic acid encoding the ASPRV1 protein.

Compounds that may be used for treating diseases associated with neutrophils include, for example, an anti-ASPRV1 antibody or an antigen-binding fragment thereof, indinavir or an analogue thereof, a siRNA, an antisense, peptides, peptidomimetics, organic molecules and other small-molecule inhibitors.

Suitable anti-ASPRV1 antibody or antigen-binding fragment thereof may be identified by their ability to inhibit ASPRV1 auto-cleavage, their ability to inhibit ASPRV1 dimerization, their ability to compete with indinavir, their ability to block the interaction of ASPRV1 with a ligand.

Mammals suffering or suspected of suffering from a demyelinating autoimmune disease as well as other autoimmune and inflammatory diseases involving neutrophils may benefit from such treatment. Demyelinating autoimmune diseases encompass multiple sclerosis, neuromyelitis optica spectrum disorder and acute disseminated encephalomyelitis.

In accordance with the present invention, the compound may be administered during the acute phase of the disorder. Alternatively, the compound may be administered during the chronic phase of the disorder.

For the purpose of drug development, it may be particularly useful that an antibody binds to different mammal species in order to test their activity, pharmacological property or toxicity in various animal models. Therefore, the present invention provides an antibody that is capable to bind human ASPRV1 and to an ASPRV1 protein that is at least 70% identical to human ASPRV1.

For example, the antibody or antigen-binding fragment thereof may bind to human ASPRV1 and to at least one other species selected from the group consisting of mouse ASPRV1 and primate ASPRV1.

Inhibitors of ASPRV1 may be identified by a method which may comprise a step of independently contacting an ASPRV1 protein or an active fragment thereof or a cell expressing the ASPRV1 protein or the active fragment thereof with a series of putative inhibitors the structure of which is identifiable and identifying an inhibitor that inhibits the activity of ASPRV1, that inhibits the dimerization of ASPRV1 or that inhibits the auto-cleavage of ASPRV1. Putative inhibitors may be obtained from a library of antibodies or antigen-binding fragments thereof, a library of small molecules, a library of peptides, a library of peptidomimetics, a library of compounds that may comprise a nucleic acid portion that is complementary to an ASPRV1 mRNA or cDNA or to a complement thereof.

The present invention also provides a method for identifying an inhibitor of neutrophils, the method may comprise contacting neutrophils (e.g., isolated and/or substantially purified neutrophils) with an antibody or antigen-binding fragment thereof that is capable of specific binding to the ASPRV1 protein or to a fragment thereof and assessing the activity of the neutrophils by methods known to a person skilled in the art, non-limiting exemplary embodiments of which include myeloperoxidase assays and elastase release assays. The activity of neutrophils may also be assessed by differential expression profiling.

A disease associated with neutrophils may thus be treated with the inhibitor thus identified.

The Applicant also discovered that extravascular neutrophils express the cell surface marker ICAM1 (ICAM1⁺ neutrophils), whereas those that are intravascular do not (ICAM1⁻ neutrophils).

The present invention therefore provides a method for detecting extravascular neutrophils by detecting ICAM1 at their surface. ICAM1⁺ neutrophils may therefore be isolated based on the presence of cell surface markers including ICAM1.

The present method may thus allow distinction between extravascular neutrophils and intravascular neutrophils by assessing whether they express ICAM1 at their surface or not.

### Example 1: ICAM1 Distinguishes Extra- from Intravascular Neutrophils in the CNS of EAE Mice

To study the phenotype of neutrophils in EAE, cell-surface markers (CD45, CD11b, Ly6G, ICAM1) were analyzed by flow cytometry in the spinal cord and blood of mice immunized with MOG₃₅₋₅₅ and adjuvants (i.e. complete Freund's adjuvant and pertussis toxin). Sham-immunized mice (adjuvants only) and naïve mice were used as controls. Samples were taken at day 15 post-immunization—a time point at which all mice had developed signs of EAE (mean clinical score, 1.9±0.2) and at which neutrophils were expected to be mobilized to the CNS¹⁵⁻¹⁷.

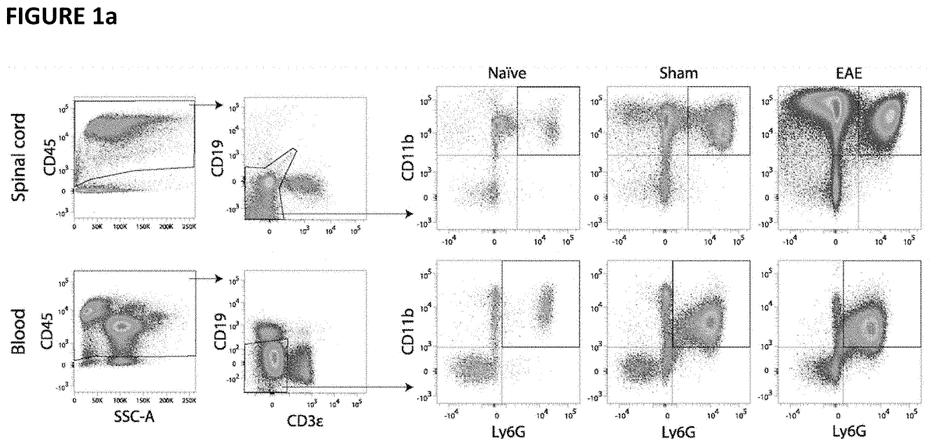

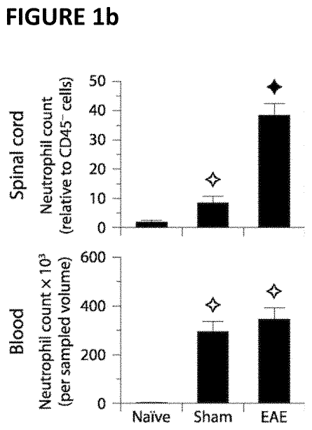

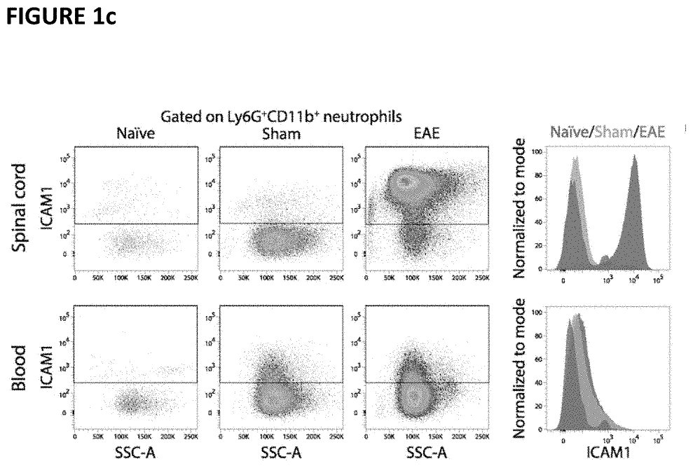

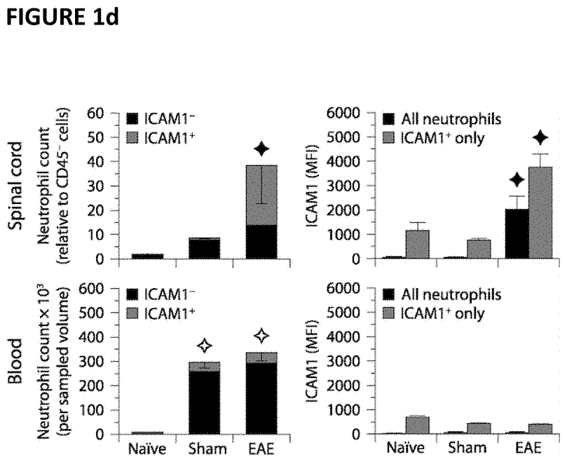

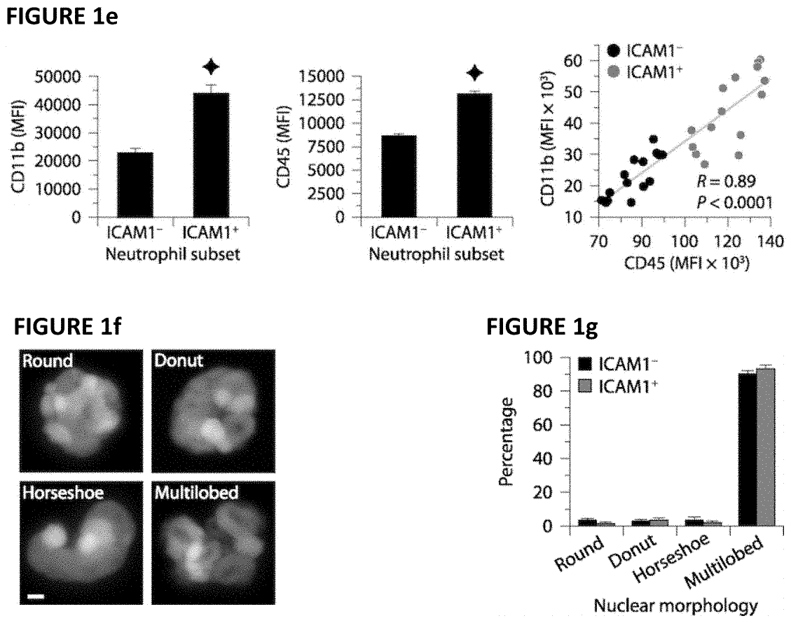

As shown in FIG. 1a-b, neutrophils (CD45⁺CD11b⁺Ly6G⁺) were expanded in the spinal cord and blood of both EAE mice and sham-immunized mice relative to naïve controls. While this cell expansion in the blood was comparable between these two groups, it was 4.3 times higher in the CNS of EAE mice relative to sham-immunized mice (FIG. 1b). These results are consistent with the prior observation that neutrophils crawl more frequently on the CNS endothelial surface upon exposure to adjuvants^{16,18,19}, but infiltrate the parenchyma only during EAE¹⁵⁻¹⁷.

Interestingly, ICAM1 was highly expressed (median fluorescence intensity, 2052±510) on a large proportion of spinal-cord neutrophils (64%±9) in EAE, but not in sham (FIG. 1c-d, upper panels). ICAM1 was generally not expressed on blood neutrophils, except at low levels (median fluorescence intensity, 84±5) and on a small proportion (13%±3) in EAE and sham (FIG. 1c-d, lower panels). In the spinal cord, ICAM1⁺ neutrophils expressed higher levels of CD11b and CD45 than did ICAM1⁻ neutrophils (FIG. 1e). Both populations were identical on the basis of their forward and side scatter properties (data not shown) and nuclear morphologies (FIG. 1f, g). Similar observations were made at other time points (e.g. day 12, 24) and in brain samples (data not shown).

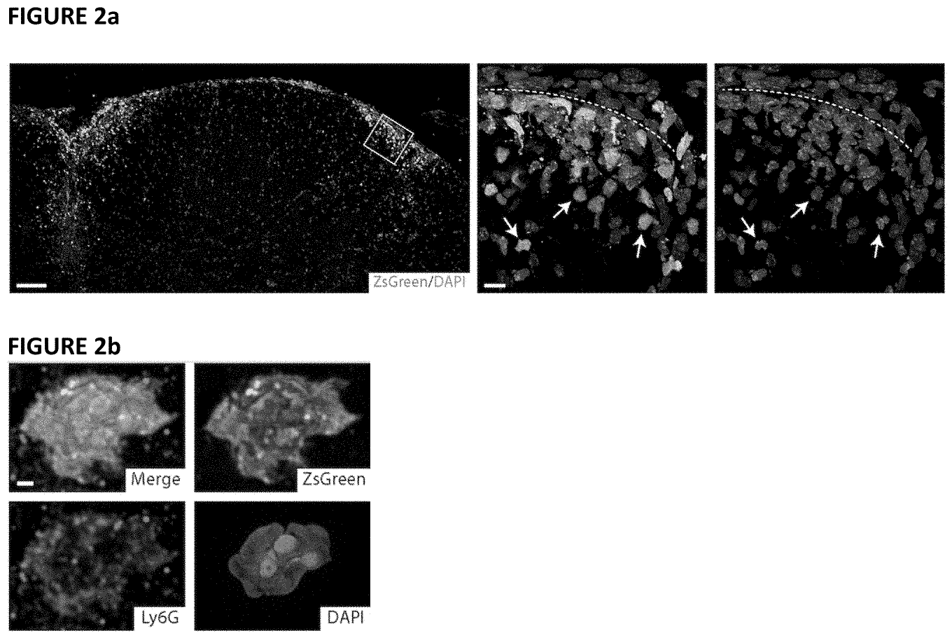

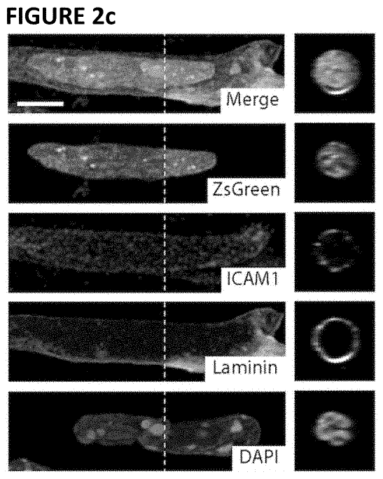

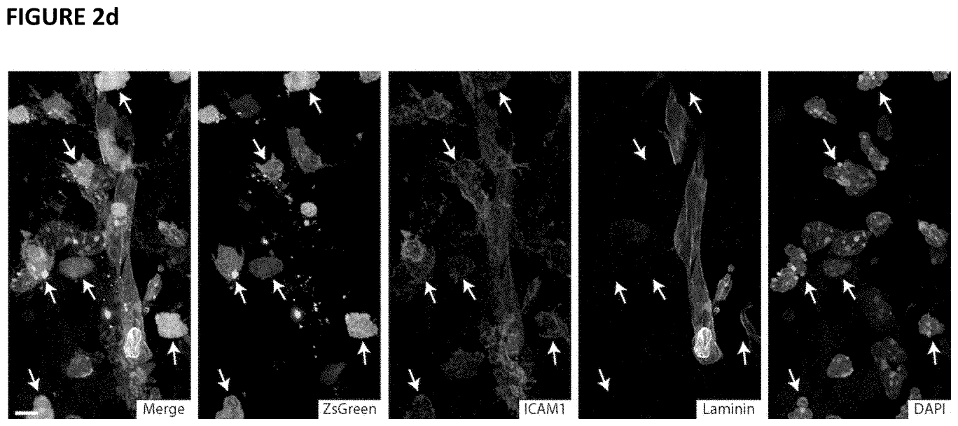

To facilitate the anatomical localization of ICAM1⁺ and ICAM1⁻ neutrophils, we generated reporter mice with green fluorescent neutrophils by crossing heterozygous Catchup mice²⁰ (expressing Cre recombinase under the neutrophil-specific Ly6G promoter) with Ai6 mice²¹ (expressing ZsGreen fluorescent protein permanently upon Cre activity). Spinal cords were collected for confocal imaging 15 days after EAE induction. In general, ZsGreen⁺ neutrophils (with characteristic multilobed nuclei) were concentrated in inflammatory foci near the central canal and in meningeal and submeningeal areas of the spinal cord (FIG. 2a). These cells were confirmed to be neutrophils by co-localization of ZsGreen with Ly6G (FIG. 2b). ICAM1 was detected on capillaries, but not on intravascular neutrophils that exhibited the rod-shaped morphology typical of crawling leukocytes²² (FIG. 2c). In contrast, ICAM1 was detected on the vast majority (>90%) of extravascular neutrophils in the meninges and parenchyma (FIG. 2d). Neutrophils were also observed in the vasculature of naïve and sham-treated mice (where they were negative for ICAM1), but never in the parenchyma, as previously reported^{18,19}.

We conclude the existence of two populations of neutrophils in the CNS of EAE mice: one patrols the CNS vasculature by crawling on its inner surface and is characterized by the absence or very low levels of ICAM1; the other, more abundant, is recruited into the meninges and parenchyma by an antigen-driven mechanism and is characterized by strong expression of ICAM1 and higher levels of CD11b and CD45. This phenotype suggests a state of increased activation²³²⁴. We propose that circulating ICAM1⁻ neutrophils (non-activated) have the potential to transmigrate across the CNS vasculature and to mature into ICAM1⁺ neutrophils (activated) that contribute to EAE development.

### Example 2: ICAM1⁺ Neutrophils have a Distinctive Transcriptional Profile Revealing a Potential for Antigen Presentation and Immunostimulation

To compare the transcriptomes of ICAM1⁺ and ICAM1⁻ neutrophils (CD45^{hi}CD11b⁺CD11c⁻Ly6G⁺), we purified these cells by FACS from the spinal cord of EAE mice at day 15 post-immunization. For comparison, we simultaneously purified two other subsets of myeloid cells: macrophages (CD45^{hi}CD11b⁻CD11c⁻Ly6G⁻) and DCs (CD45^{hi}CD11b⁺CD11c⁺Ly6G⁻). RNA was analyzed in biological duplicate using Affymetrix GeneChip Mouse Gene 2.0 ST arrays interrogating 28,137 coding transcripts. Data were then normalized and quantified using the RMA algorithm.

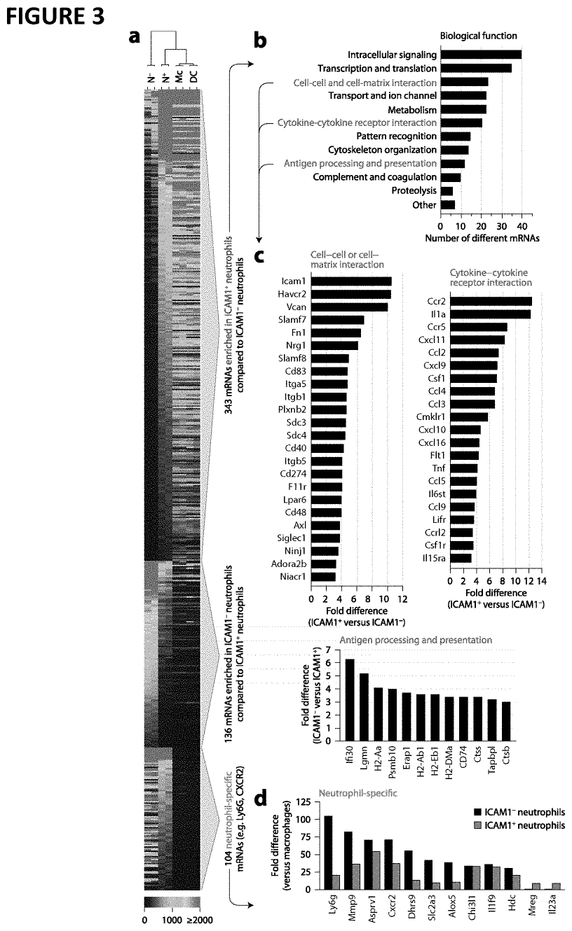

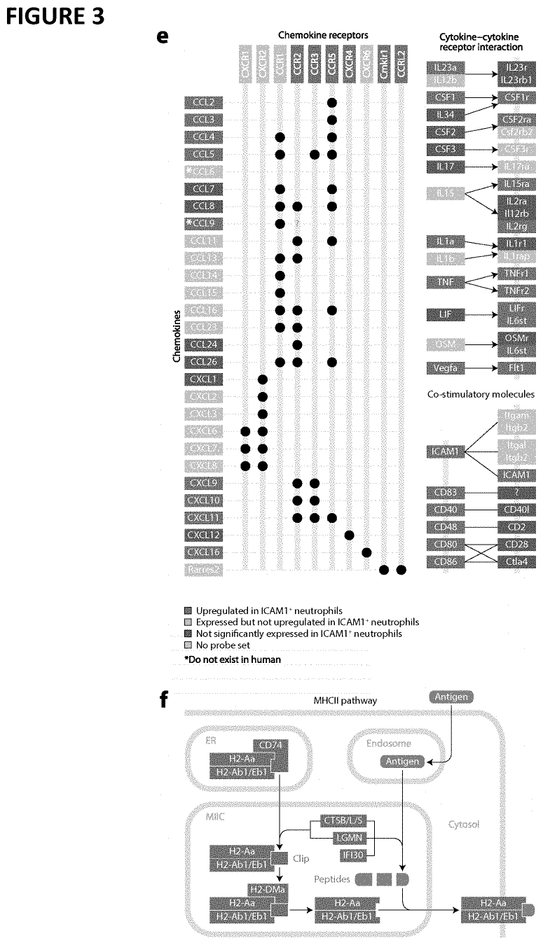

Unsupervised hierarchical clustering revealed that the transcriptome of ICAM1⁺ neutrophils was substantially different from that of ICAM1⁻ neutrophils (FIG. 3a). Among the 479 genes that were differentially expressed between these cells according to stringent criteria (fold change 3, mean hybridization signal 200 in at least one subset, P-value 0.05), 343 were upregulated and 136 were downregulated in ICAM1⁺ neutrophils (FIG. 3a). Hence, neutrophils are functionally plastic in EAE: after extravasation into the inflamed CNS, they acquire distinct properties through a substantial transcriptional remodeling.

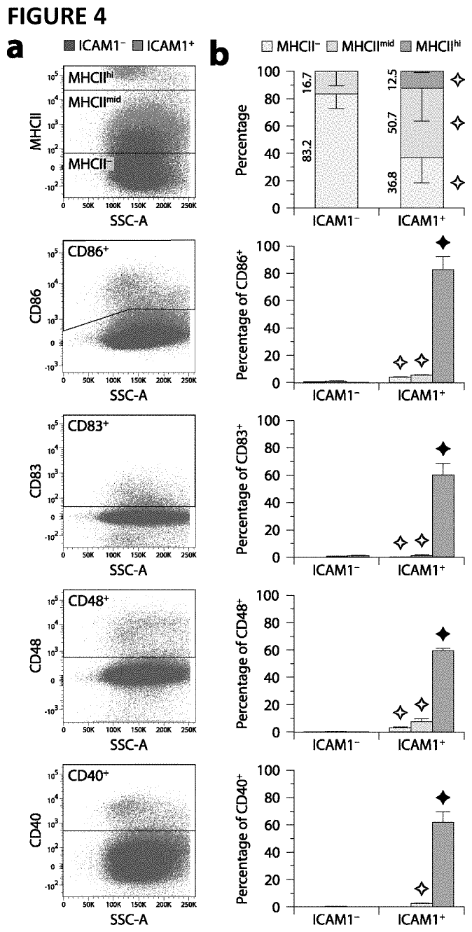

Hierarchical clustering also showed that ICAM1⁺ neutrophils were more similar to macrophages and DCs than ICAM1⁻ neutrophils were (see dendrogram in FIG. 3a). Of the 343 genes that were upregulated in ICAM1⁺ neutrophils, 328 (96%) were also expressed by macrophages and/or DCs at similar or higher levels. Further, 232 (68%) had a known function and could be manually divided into 12 functional categories, including cell-cell or cell-matrix interaction, cytokine-cytokine receptor interaction and antigen presentation (FIG. 3b). These three latter categories also stood out as over-represented when the classification was done with software tools (KEGG and Ingenuity Pathway Analysis, P<0.0001). Notably, they included many genes coding for cytokines (e.g. IL-1α, CSF1), leukocyte chemoattractants (e.g. CXCL9-11, CCL2-5), cell-surface receptors (e.g. CCR2, CCR5, HAVCR2, NIACR) and adhesion molecules (e.g. VCAN) (FIG. 3c). Remarkably, ICAM1⁺ neutrophils upregulated genes at every step along the antigen-processing and -presentation pathway (FIG. 3c): proteases that intracellularly process protein antigens (e.g. LGMN, IF130, CTSB); subunits of the immunoproteasome (e.g. PSMB10); chaperones necessary for MHCII complex formation (e.g. CD74/li, H2-DMa); MHCII subunits themselves (H2-Aa, H2-Ab1, H2-Eb1); and co-stimulatory molecules (e.g. CD40, CD48, CD83, CD86). By flow cytometry, the presence of MHCII and co-stimulatory molecules was confirmed on a considerable proportion of ICAM1⁺ neutrophils: 12.5% expressed high levels of MHCII with co-stimulatory molecules, 50.7% expressed moderate levels of MHCII, and 36.8% were MHCII-negative (FIG. 4). These results indicate that neutrophils, after infiltrating the CNS, acquire macrophage/DC properties, including the potential ability to secrete immunostimulatory factors and present antigen to lymphocytes (FIG. 3e, f).

Another 104 genes were predominantly expressed in neutrophils and not or weakly in the other myeloid cells (FIG. 3a). Those that were expressed in both neutrophil subsets included genes coding for well-known neutrophil markers (e.g. Ly6G, CXCR2, MMP9), the recently discovered neutrophil cytokine IL-36γ²⁵ (also called IL1F9), the enzyme histidine decarboxylase (HDC) that synthesizes histamine, and new potential neutrophil markers with unclear functions (e.g. ASPRV1, Chi3l1) (FIG. 3d). Thirteen of these 104 genes, notably melanoregulin (MREG) and IL-23a (FIG. 3d), were enriched >3-fold in ICAM1⁺ neutrophils compared to ICAM1⁻ neutrophils. Thus, it appears that neutrophils are equipped with a specific set of molecules allowing them to execute unique functions in EAE, in addition to roles redundant with those of macrophages and DCs.

### Example 3: ICAM1⁺ Neutrophils do not Differentiate into Ly6G⁻ Monocytic Cells

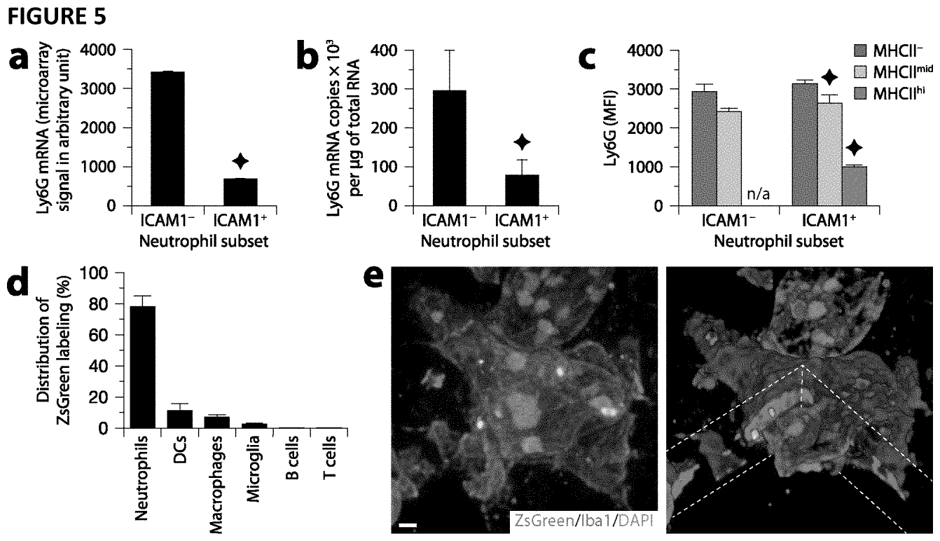

Our microarray and RT-qPCR data revealed that Ly6G mRNA was ˜5 times less abundant in ICAM1⁺ neutrophils compared to ICAM1⁻ neutrophils (FIGS. 5a, b). This observation prompted us to examine whether Ly6G was uniformly distributed among the three subpopulations of neutrophils defined by the levels of MHCII. Flow cytometric analysis revealed that Ly6G expression was roughly similar on MHCII⁻ and MHCII^{mid} neutrophils, but threefold lower on MHCII^{hi} neutrophils (FIG. 5c).

An intriguing question arises as to whether MHCII^{hi}Ly6G^{lo} neutrophils ultimately go on to lose Ly6G completely. If this were correct, then gating our samples on Ly6G as a neutrophil marker would lead to underestimating the number of infiltrating neutrophils and to exclude a potentially important neutrophil subset. The possibility that neutrophils become negative for Ly6G is supported by an independent study showing that neutrophils can differentiate into Ly6G⁻ monocytic cells under inflammatory conditions in vivo²⁶. To test this possibility in EAE, we permanently labeled neutrophils using the Catchup×Ai6 model (described above) and analyzed them by flow cytometry. Approximately 80% of all Ly6G⁺ neutrophils from the spinal cord of EAE mice expressed ZsGreen. Curiously, Ly6G⁺ neutrophils accounted for 78% of the ZsGreen⁺ population, while the remaining 22% were other myeloid phagocytes: DCs, macrophages and microglia (FIG. 5d).

To determine whether this latter result was real differentiation or simply attributable to engulfment of ZsGreen⁺ neutrophil debris, we examined spinal cord sections immunostained for Iba1 (also known as Aif1), a marker of microglia and other myeloid phagocytes²⁷. We found that virtually all cells double positive for ZsGreen and Iba1 were actually cells containing ZsGreen⁺ debris in their cytoplasm (FIG. 5e). Yet, we did see a few cells that were truly double positive with ZsGreen throughout the cytoplasm (data not shown); however, these cells exhibited a multilobed nucleus, expressed lower levels of Iba1 than other myeloid cells (consistent with our microarray data revealing Iba1 expression in ICAM1⁺ neutrophils), and accounted for only a minor fraction of ZsGreen⁺ cells (<1%).

Thus, we conclude that ICAM1⁺ neutrophils decrease, but do not lose, their expression of Ly6G during migration into the CNS parenchyma; there, they adopt some macrophage characteristics, but do not differentiate into a different myeloid cell type. The diminution of Ly6G expression could still reduce the number of cells estimated by immunofluorescence, due to the lower sensitivity of the technique. Furthermore, flow cytometric analysis of neutrophils in the Catchup×Ai6 model must be done with caution, as a significant proportion of ZsGreen⁺ cells may be contamination with other myeloid phagocytes that have internalized remnants of dead neutrophils, a phenomenon also observed in stroke²⁸.

### Example 4: ICAM1⁺ Neutrophils Form Immunological Synapses with T and B Cells

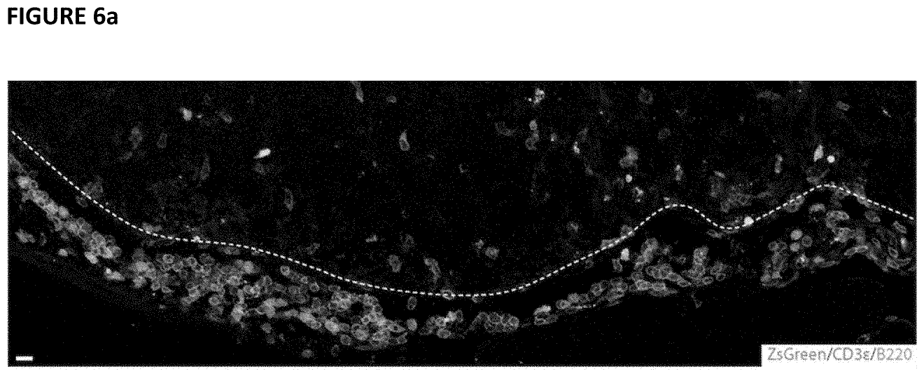

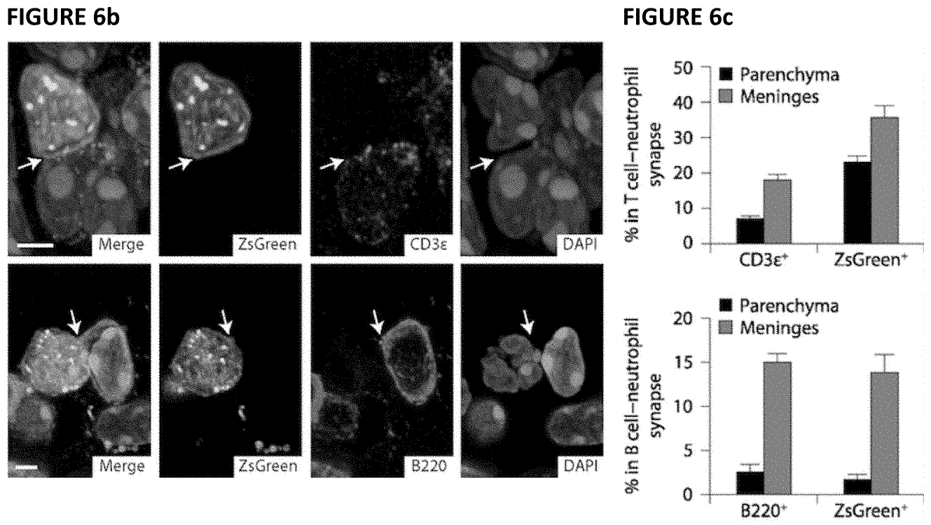

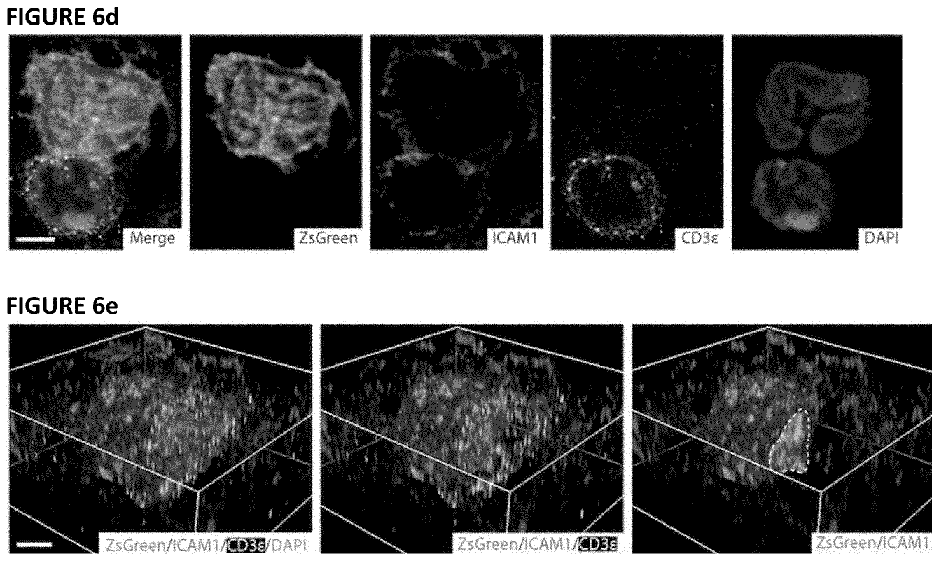

Our microarray results indicate that infiltrating neutrophils upregulate proteins known to be important for physical and functional interactions with lymphocytes (e.g. ICAM1, MHCII and co-stimulatory molecules) (FIG. 3, 4). To determine whether such interactions occur in our model, spinal cord sections from Catchup×Ai6 mice with EAE were stained for CD3ε and B220, then the number of neutrophils physically contacting lymphocytes was estimated. FIG. 6a gives an overview of one such section, where neutrophils and T cells were localized both in the parenchyma and surrounding meninges, whereas most B cells were restricted to the meninges. At higher magnification, we observed, across the tissue, many neutrophils that were juxtaposed to T and B cells (FIG. 6b). In all individuals studied (four), these contacts were frequent: in the meninges, for example, ˜36% of ZsGreen⁺ neutrophils contacted CD3ε⁺ T cells, while another ˜14% contacted B220⁺ B cells (FIG. 6c).

T cells can contact APCs to form immunological synapses, which are characterized by a ring of ICAM1 surrounding a central zone devoid of ICAM1²⁹. The occurrence of immunological synapses in CNS tissue has only been thus far documented between cytotoxic T cells and astrocytes during infection^{30,31} To investigate the fine-scale structure of neutrophil-T cell doublets, we performed super-resolution microscopy by stimulated emission-depletion (STED) with a pulsed depletion beam and time-gated detection (FIG. 6d). 3D reconstructions revealed the presence of a zone devoid of ICAM1 on neutrophils at the plane of contact with T cells (FIG. 6e). To our knowledge, this represents the first in vivo evidence that neutrophils form immunological synapses with T cells. Our observations suggest that neutrophils recruited to the mouse CNS during EAE can physically engage with T and B cells and form immunological synapses with T cells, reminiscent of antigen presentation.

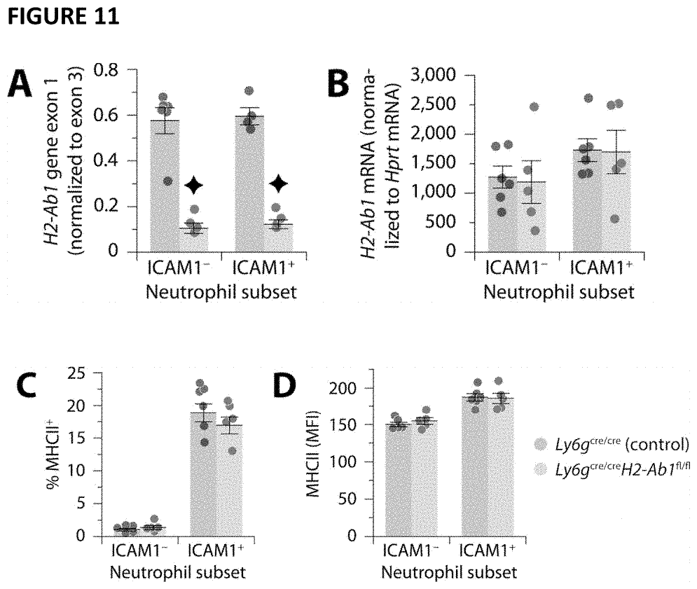

To directly test the importance of the antigen presenting capability of neutrophils in EAE, we crossed Catchup mice to a conditional null strain for H2-Ab1³² (the only extant MHCII-chain allele in C57BL/6 mice and among those genes upregulated in ICAM1⁺ neutrophils) in order to abolish MHCII expression specifically in neutrophils. Efficient (>80%) gene deletion was confirmed in genomic DNA obtained from spinal-cord neutrophils (FIG. 11A), yet, perplexingly, H2-Ab1 mRNA and protein were unchanged (FIG. 11B-D). These observations, together with the fact that bone marrow neutrophils express H2-Ab1 mRNA (data not shown), lead us to propose that H2-Ab1 mRNA is expressed early in neutrophil development, before Cre can delete the gene. Thus, consistent with previous findings²⁰, proteins such as H2-Ab1 that are regulated at the translational level from pre-formed mRNAs cannot be studied in the Catchup model.

### Example 5: ASPRV1 is Specifically Expressed by Neutrophils in Mouse and Human, and Correlates with Neutrophil Infiltration in Demyelinating Autoimmune Diseases

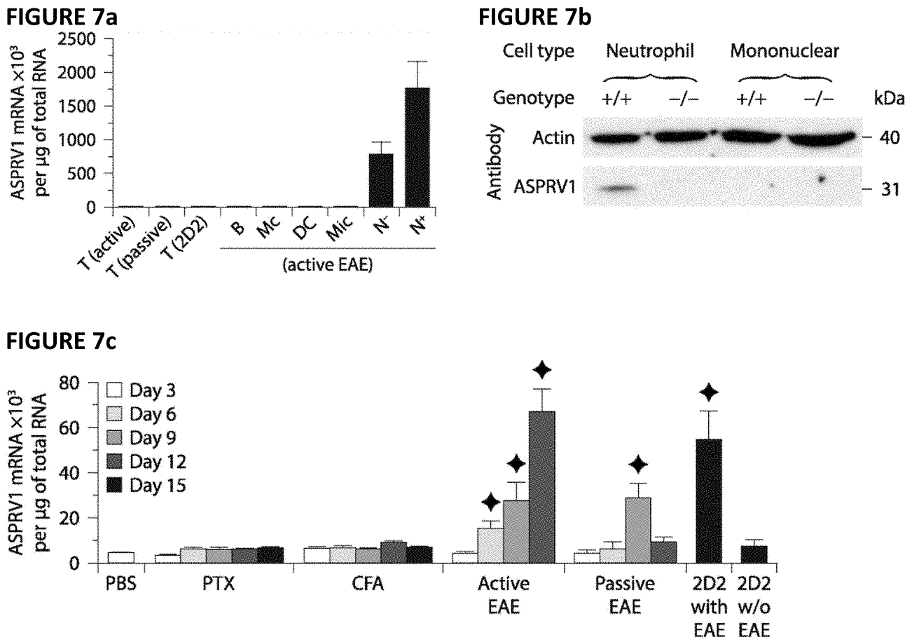

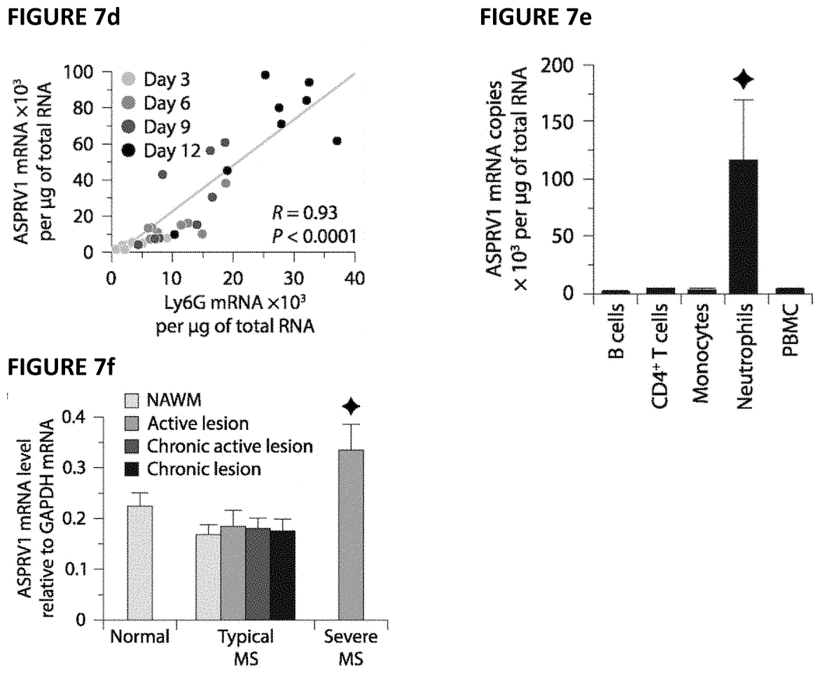

Seeking to discover a unique function of neutrophils in EAE, we chose to follow up on ASPRV1, because it was the most highly expressed neutrophil-specific gene with a human homolog that had not yet been studied in the immune or nervous system, and because it encodes a little-known protease that could play a novel effector role in inflammation. By RT-qPCR, we found that ASPRV1 mRNA was indeed expressed in neutrophils (ICAM1⁺ and ICAM1⁻) extracted by FACS from EAE spinal cords, but not in any other immune cell types tested (FIG. 7a). ASPRV1 protein was also expressed in neutrophils from mouse bone marrow, as revealed by Western blotting with the same antibody used to first detect ASPRV1 in skin⁹ (FIG. 7b). Specificity was confirmed by the absence of signal in mononuclear leukocytes and ASPRV1^{-/-} neutrophils (FIG. 7b). In whole spinal cord extracts, ASPRV1 mRNA was barely detectable in the absence of inflammation and upon adjuvant injection (FIG. 7c); however, it was highly expressed during EAE and strongly correlated with Ly6G mRNA (FIG. 7d). The abundance of ASPRV1 mRNA was remarkable, i.e. higher than or equal to Ly6G mRNA, both in spinal cord extracts and purified neutrophils (FIG. 7d).

To translate our findings to human, we analyzed, by RT-qPCR, blood fractions and post-mortem brain samples from people with or without MS. ASPRV1 mRNA was abundant in blood neutrophils in the steady state, but not in B cells, T cells, monocytes or unfractionated mononuclear cells (FIG. 7e). Further, ASPRV1 mRNA was detected in higher amounts in brain lesions from patients with severe MS compared to both normal-appearing white matter and brain lesions from patients with typical forms of MS (FIG. 7f). Therefore, our results demonstrate that ASPRV1 is a neutrophil-specific protein in the immune system and can serve as a neutrophil marker in the nervous system both in the mouse and human.

### Example 6: ASPRV is Required for the Chronic Phase of a B Cell-Dependent EAE Model

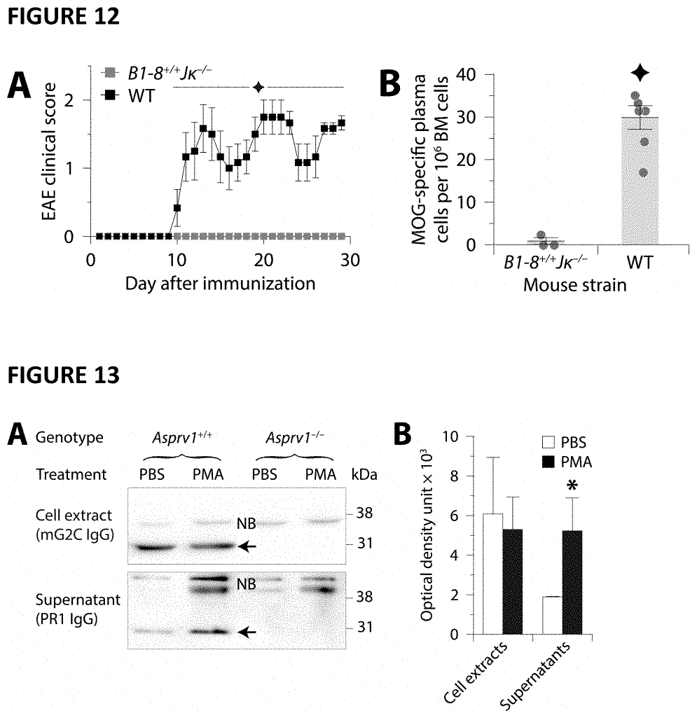

To determine whether ASPRV1 contributes to EAE, we immunized ASPRV1-deficient and wild-type mice with two different antigens: the standard MOG₃₅₋₅₅ peptide and bMOG. bMOG is a novel “humanized” mouse MOG₁₋₁₂₅ protein that bears the S42P mutation abolishing the immunodominant T-cell epitope (amino acids 35-55). bMOG can still induce EAE with prominent neutrophilic infiltration, but contrary to MOG peptide³³, it acts through a B cell-dependent mechanism. This was demonstrated in B1-8^{+/+}Jκ^{-/-} mice expressing a single B cell receptor to an irrelevant antigen, which mice did not develop EAE in response to bMOG (FIG. 12). Therefore, compared to MOG₃₅₋₅₅, bMOG induces a form of EAE that is more similar to MS as it involves pathogenic B cells.

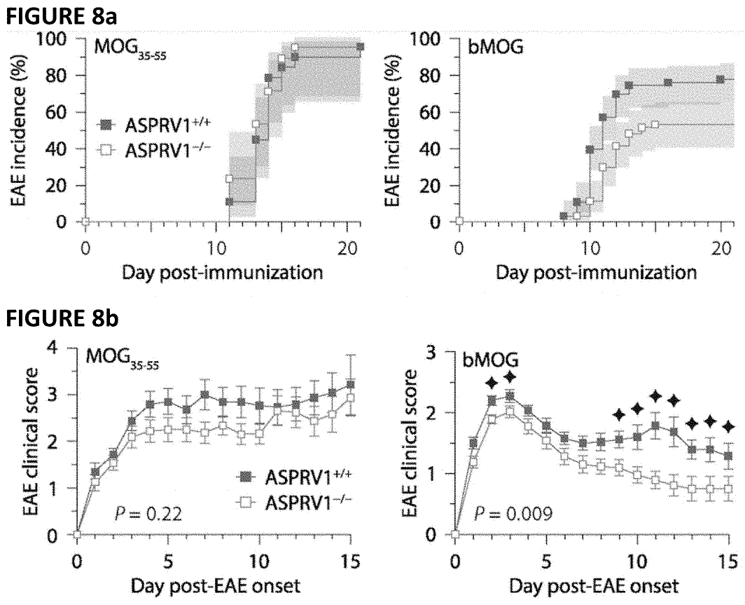

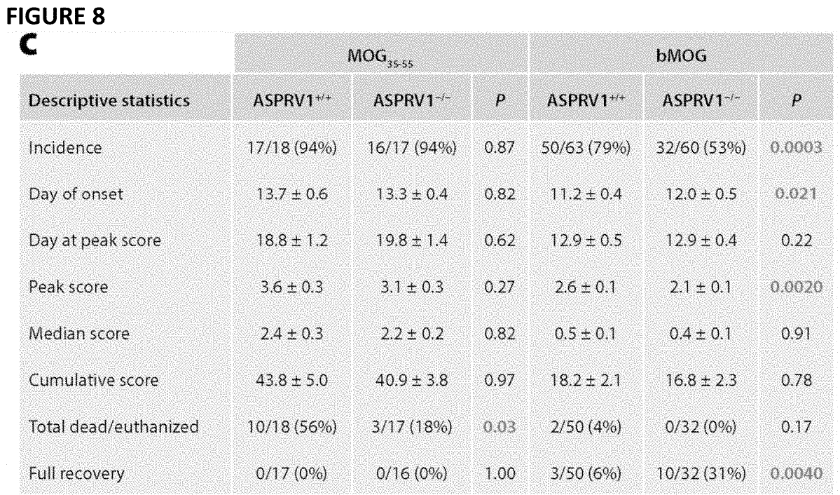

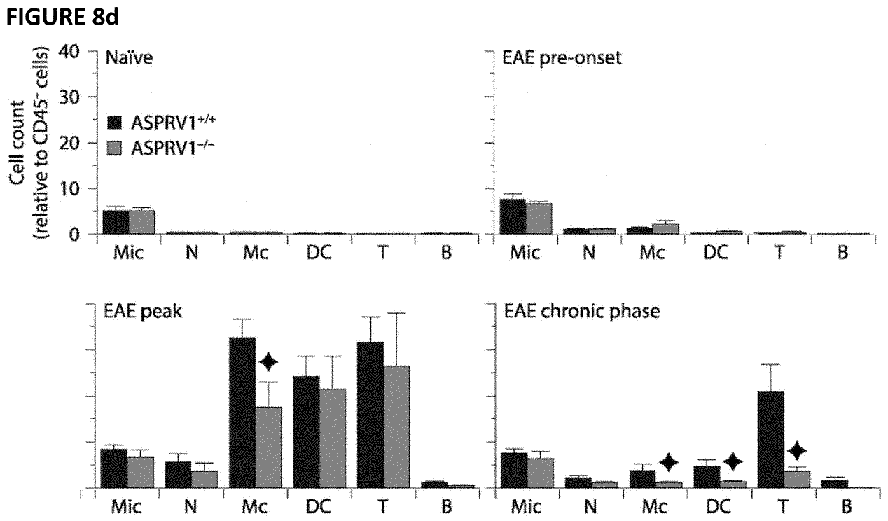

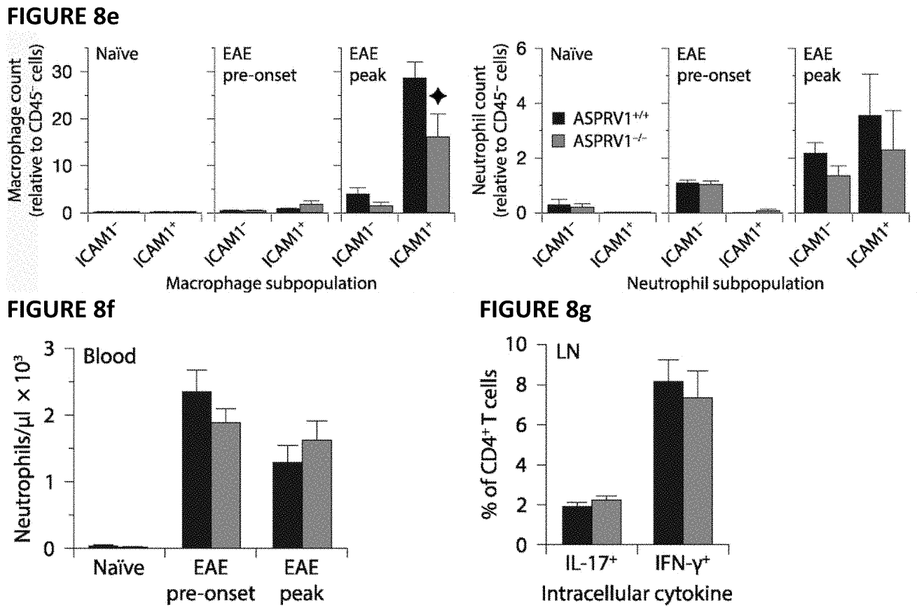

After immunization with MOG₃₅₋₅₅, no difference was observed in EAE incidence and severity between ASPRV-1^{-/-} and wild-type mice (FIG. 8a-c). In contrast, with bMOG, EAE incidence and acute phase were slightly reduced in the absence of ASPRV1 (FIG. 8a-c). More importantly, the chronic phase was blunted in ASPRV-1^{-/-} mice; after a recovery phase culminating around day 7 post-onset, wild-type mice experienced a relapse, whereas ASPRV-1^{-/-} mice continued to recover, so that 31% of them showed no more clinical signs by the end of experimentation (FIG. 8b, c).

To corroborate and explain these symptomatic differences, we performed flow cytometric analysis on spinal cords at different phases of bMOG-induced EAE. At pre-onset (day 8 post-immunization), only neutrophils and macrophages had begun to accumulate (P≤0.0012 with respect to naïves), but to a similar extent in ASPRV1^{-/-} and wild-type mice (FIG. 8d). At the peak of EAE (day 13 post-induction), all types of infiltrating leukocytes reached their maximal numbers; among them, only macrophages were less numerous in the knockouts (by 46%) (FIG. 8d). More precisely, this reduction predominantly affected the ICAM1⁺ subpopulation of macrophages (FIG. 8e), which, by analogy with neutrophils, may be presumed to be extravasated. No abnormality was noticed in the state of activation of either ICAM1⁺ macrophages or neutrophils, as inferred from their surface levels of CD45, CD11b, ICAM1, MHCII and CD86 (data not shown). By the chronic phase (day 21 post-immunization), many leukocytes (neutrophils, macrophages, DCs) had left the spinal cord of both mouse strains, leaving behind a sizeable population of T cells; in ASPRV-1^{-/-} mice, even fewer cells remained, and the number of T cells was only one-sixth that of wild-type mice (FIG. 8d).

The lower sensitivity of ASPRV-1^{-/-} mice to bMOG was not attributable to a defect in neutrophil migration, because no substantial intergenotype difference was seen either in the number of ICAM1⁺ neutrophils that had infiltrated the CNS tissue at the peak of EAE (FIG. 8e) or in the number of ICAM1⁻ neutrophils crawling inside the CNS vasculature (FIG. 8e) or circulating in blood (FIG. 8f). Nor was this lower sensitivity attributable to a defect in T cell priming, because the proportion and encephalitogenic capacity of Tₕ1 and Tₕ17 cells in draining lymph nodes did not differ between the genotypes (FIG. 8g), which is consistent with a previous study showing that neutrophil depletion does not affect the priming of encephalitogenic T cells³⁴. Finally, this phenotype was only associated to ASPRV1 and not observed in mice lacking other neutrophil-specific proteins (IL-36y and MREG; incidence, P≥0.85; day of onset, P>0.55; severity over time, P≥0.35; n>10-12 per group). Overall, our results demonstrate that: 1) ASPRV1 is required both to reach maximum disease severity and to sustain chronic inflammation in response to bMOG; and 2) ASPRV1 does not significantly affect neutrophil mobilization from bone marrow, adhesion/crawling on the CNS vasculature, or extravasation into meningeal and submeningeal compartments, which is consistent with the normal response of ASPRV1^{-/-} mice to MOG₃₅₋₅₅. Therefore, neutrophils exert, most likely in the CNS, a proinflammatory effect via ASPRV1 that is crucial for perpetuating inflammation specifically in a B-cell-dependent form of EAE.

### Example 7: ASPRV1 is Secreted from Neutrophils

Bone marrow cells were collected by flushing out femurs with 10 mL of HBSS (Wisent Bioproducts), filtered through 70-μm mesh, resuspended in HBSS containing 45% Percoll (GE Healthcare), and centrifuged at 1,600×g for 30 min over a four-level Percoll density gradient (45%, 55%, 65%, 81%). Neutrophils were harvested at the 65-81% interphase, purified with a negative selection kit (EasySep Mouse Neutrophil Enrichment kit, Stemcell Technologies), and incubated at 37° C. for 30 min at a concentration of 12.5 millions/ml in HBSS containing or not 100 nM phorbol 12-myristate 13-acetate (PMA). After centrifugation, cells were lysed in Laemmli buffer, while supernatants were concentrated 6 times using Vivaspin 5000 MWCO ultrafiltration columns. Total proteins equivalent to 2 million neutrophils were separated on a 15% SDS-polyacrylamide gel and transferred to either a polyvinylidene difluoride membrane (cell extracts) or a nitrocellulose membrane (supernatants). Western blotting for ASPRV1 was performed as described previously (Hawkins et al., 2017).

Our results demonstrate that ASPRV1 is secreted from neutrophils (FIG. 13), providing evidence that ASPRV1 could be therapeutically targeted with antibodies. The secreted short form of mouse ASPRV1 is represented by SEQ ID NO.:5 and the corresponding human form is represented by SEQ ID NO.:6.

### DISCUSSION

The importance of neutrophils for EAE and the mechanism of their recruitment into the CNS have already been extensively investigated⁶, but their phenotype and functions are largely unknown. For the first time, the present study: 1) identifies a surface marker (ICAM1) that distinguishes extravascular from intravascular neutrophils; 2) identifies a neutrophil-specific marker (ASPRV1) applicable in both mouse and human within the immune and nervous systems; 3) deciphers the transcriptomic changes undergone by neutrophils after extravasation, hence revealing functional plasticity (e.g. acquisition of macrophage-like immunostimulatory and antigen presentation properties); 4) shows in vivo that neutrophils make intimate physical contacts with T and B lymphocytes at the site of inflammation; 5) specifies the perpetuation of chronic inflammation as one context-dependent function of neutrophils; 6) ascribes an extra-epithelial role to ASPRV1 as a proinflammatory effector; and 7) introduces a novel humanized myelin antigen (bMOG) that can be used to induce a B cell-dependent form of EAE and to study neutrophil-B cell interactions.

ICAM1 as a Marker for Extravasated Neutrophils

Under healthy conditions, neutrophils continually crawl within the CNS vasculature, but do not cross the blood-brain barrier; in EAE, neutrophils become much more numerous, both continuing to patrol within blood vessels and beginning to extravasate into the meninges and parenchyma¹. Previously, when analyzing CNS samples by flow cytometry, the patrolling and infiltrating neutrophil populations could not be separated. Recent studies have reported ICAM1 expression by neutrophils under certain conditions of activation³⁵⁻³⁷; we show here that the great majority of extravasated neutrophils in EAE express ICAM1, but not patrolling neutrophils. By allowing a better adhesion between leukocytes (e.g. via LFA1), ICAM1 facilitates cell-cell interactions such as the formation of immunological synapses²⁹. Our results suggest that neutrophils act, at least in part, in a contact-dependent manner in demyelination, and provide a surface marker for the study of neutrophil subpopulations.

ASPRV1 as a Neutrophil-Specific Marker in Mouse and Human

The Ly6G antigen conclusively identifies neutrophils in mice, but no such marker exists in human. Those that exist (e.g. myeloperoxidase, neutrophil elastase, CD16b and CD66b) can also be expressed by other myeloid cells and, thus, are not fully specific to neutrophils. This complicates the identification of neutrophils in human tissues (e.g. CNS), since these cells can adopt a macrophage-like phenotype and since their nucleus can be more compacted and thus difficult to recognize by conventional microscopy. We propose the use of ASPRV1 as a specific marker for neutrophils in human and mouse tissue samples, with the added benefit of facilitating translation of results from animal models.

Antigen-Presenting Function of Neutrophils

Neutrophils in culture can form immunological synapses^{38,39} and present antigen to T cells⁴⁰⁻⁴³, yet the relevance of these non-classical APCs remains to be shown in vivo. We report here that neutrophils prepare for being APCs at an early stage of development by pre-forming MHCII mRNA, which can later be translated at the site of inflammation. We also provide the first in vivo evidence for close contacts between neutrophils and T cells within EAE lesions with a redistribution of cell-surface ICAM1. Several types of immunological synapses have been described ex vivo²⁹, but more characterization is required to determine to which category the synapses we observed in EAE belong. Since current genetic models cannot specifically delete MHCII in neutrophils, new strategies will be needed to confirm the importance of these synapses. For instance, neutrophils could enhance the anti-MOG Tₕ17 response or contribute to or restrain epitope spreading by ingesting degraded myelin and presenting new epitopes to T cells in either a positive or negative regulatory manner.

ASPRV1-Dependent Function of Neutrophils

The function of ASPRV1 is only known in the context of stratified epithelia (e.g. the stratum corneum of the skin), where it processes profilaggrin and enhances skin hydration,^{8,9,11,12}. We have now found that ASPRV1 is also expressed in the immune system solely in neutrophils. However, the substrate of ASPRV1 in this new context is unknown, as profilaggrin is not expressed in neutrophils (according to our microarray data) and has not been reported in the immune and nervous systems. Since ASPRV1 was ostensibly acquired from a retrotransposon sometime around the advent of mammals⁴⁴, the selective expression of ASPRV1 in neutrophils may be associated to a mammal-specific function of neutrophils. The exact substrate of ASPRV1 in neutrophils remains to be identified. However, we can conclude that an ASPRV1-dependent function of neutrophils leads to the recruitment of macrophages and promotes chronic inflammation in EAE.

bMOG as a New Antigen for EAE Induction

B cells contribute to MS and NMOSD in two major ways: by secreting autoantibodies and by stimulating the development of autoreactive Tₕ cells via antigen presentation and secretion of cytokines (e.g. IL-6, GM-CSF and IL-15)⁴⁵. Similarly, B cells are essential for the development of EAE when induced with either bMOG, as demonstrated herein, or human MOG protein⁴⁶. However, in the traditional MOG₃₅₋₅₅ model, B cells play no such role, instead acting as beneficial, regulatory cells, since depleting them exacerbates the symptoms^{14,47-56}. This is possibly because DCs and macrophages can only present the immunodominant MOG₃₅₋₅₅ epitope to T cells, whereas B cells can process and present several epitopes, thereby mounting a more diversified response; by mutating the 35-55 epitope in bMOG, B cells become necessary APCs for T cell activation. The advantages of using bMOG are twofold: first, compared to the traditional MOG peptide, bMOG represents an improved model for investigating B cell-dependent responses, which is especially important given the recent success of B cell depletion therapy for MS⁴⁵; second, compared to human MOG, bMOG differs from mouse MOG by only a single amino acid, thus excluding possible confounding effects of multiple mutations. Use of bMOG should help mechanistic studies of how the immune response can drift from foreign to self-epitopes.

### CONCLUSION

Long considered a black sheep in the field of autoimmune demyelination, neutrophils should rather be seen as an essential component of a cooperative network of immune cells interacting chemically and physically with one another. By linking an ASPRV1-dependent function of neutrophils to the progression of a B-cell-dependent form of EAE, our study suggests that neutrophils could be engaged in a unique way in some forms and stages of demyelinating diseases. More specifically, our study points to a possible role for neutrophils as downstream effectors of B cells in autoimmunity. Furthermore, our work provides new tools (markers, transcriptomic profiles, transgenic models, bMOG) to explore the many facets of neutrophils. In particular, ASPRV1 could prove a useful biomarker for identifying the presence of neutrophils in biological samples for basic research and clinical diagnosis. Future challenges include finding the substrate(s) of ASPRV1 in the immune system, explaining why neutrophils exert a differential effect in B cell-dependent EAE, and determining whether ASPRV1 could be targeted for the treatment of NMOSD, some forms of MS and other immune disorders.

Methods

Human Brain Samples

Snap-frozen post-mortem CNS samples from MS and NMOSD patients and controls were obtained from the Multiple Sclerosis Society Tissue Bank at the Imperial College London. Additional samples from a patient with severe rebound MS activity after natalizumab withdrawal⁵⁷ was obtained from the University of Montreal Hospital Research Center. Biopsies were classified as acute active lesion (stage I and 2), chronic active lesion (stage 3 and 4), chronic inactive lesion (stage 5) or normal appearing white matter, as described previously⁵⁸. Frozen sections were cut with a cryostat to obtain 20 mg of tissue, which was homogenized in TRI-reagent (Sigma-Aldrich) and processed with the EZ-10 Spin Column Animal Total RNA Mini-prep Kit (Bio Basic) to extract RNA for RT-qPCR. Additional sections were used for histology. Our institutional research ethics committee approved this work.

Human Blood Samples

Healthy volunteers' blood was layered on top of lymphocyte separation medium (Wisent Bioproducts) and centrifuged for 20 minutes at 600×g. The top cell fraction, enriched in PBMCs, was used directly or processed with the EasySep Human CD4⁺ T Cell, B Cell or Monocyte Isolation kits; the lower fraction, enriched with granulocytes, was processed with the EasySep Direct Human Neutrophil Isolation kit, according to the manufacturer's instructions (StemCell Technologies). RNA was purified from cell fractions using Aurum Total RNA kit (Bio-Rad Laboratories).

Animals

C57BL/6, Ai6²¹ and H2-Ab1-floxed³² mice were obtained from Jackson Laboratory. ASPRV1^{-/-9}, IL-36γ²⁵, MREG^{-/-59} and Catchup²⁰ mice were derived from previously established colonies. B1-8 mice⁶⁰ with a homozygous deletion of the Jκ locus⁶¹ were generously provided by Dr. Ann Haberman (Yale University). Genotypes were confirmed by PCR using the primers listed in the Sequence Listing (Table 1). Experiments were performed under specific pathogen-free conditions on mice aged 8-10 weeks with the approval of our institutional animal protection committee.

bMOG Production

The pET-32 MOG_{tag} vector⁶² was mutated by PCR using the following primers: 5′-TACCG TCCGCCGTTTTCTCGCGTTGTTCACC-3′ and 5′-AAACGGCGGACGGTACCAGCCAACTTCCAT-3′. The resulting vector was sequenced to confirm the insertion of the S42P mutation, and used to produce bMOG according to a previously described protocol⁶³. Final concentration was set to 5 mg/ml with no detectable impurities, as measured by Bradford assay and SDS-PAGE.

EAE Induction

Mice were subcutaneously injected into both flanks with a total of 200 μl of emulsion containing either 300 μg of MOG₃₅₋₅₅ (Synpeptide) or 500 μg of bMOG (see above) dissolved in saline and mixed with an equal volume of complete Freund's adjuvant containing 500 μg of killed Mycobacterium tuberculosis H37 RA (Difco Laboratories). Mice were also intraperitoneally injected with 20 μg/kg of PTX (List Biological Laboratories) immediately and 2 days after immunization.

Evaluation of EAE Symptoms

Mice were weighed and scored daily as follows: 0, no visual sign of disease; 0.5, partial tail paralysis; 1, complete tail paralysis; 1.5, weakness in one hind limb; 2, weakness in both hind limbs; 2.5, partial hind limb paralysis; 3, complete hind limb paralysis; 3.5, partial forelimb paralysis; 4, complete forelimb paralysis; 5, dead or killed for humane reasons.

ELISpot

To detect MOG-specific plasma cells in bone marrow, 96-well plates were coated overnight at 4° C. with 0.5 μg mouse MOG₁₋₁₂₅. Wells were blocked with 1% (wt/vol) BSA in PBS, then incubated with bone marrow cells at 37° C. with 5% CO₂. Spots were detected using a goat alkaline phosphatase-conjugated anti-mouse IgG antibody (MABTECH) and 5-bromo-4-chloro-3-indolyl-phosphate substrate (Sigma-Aldrich), then counted under a dissection microscope.

Flow Cytometry

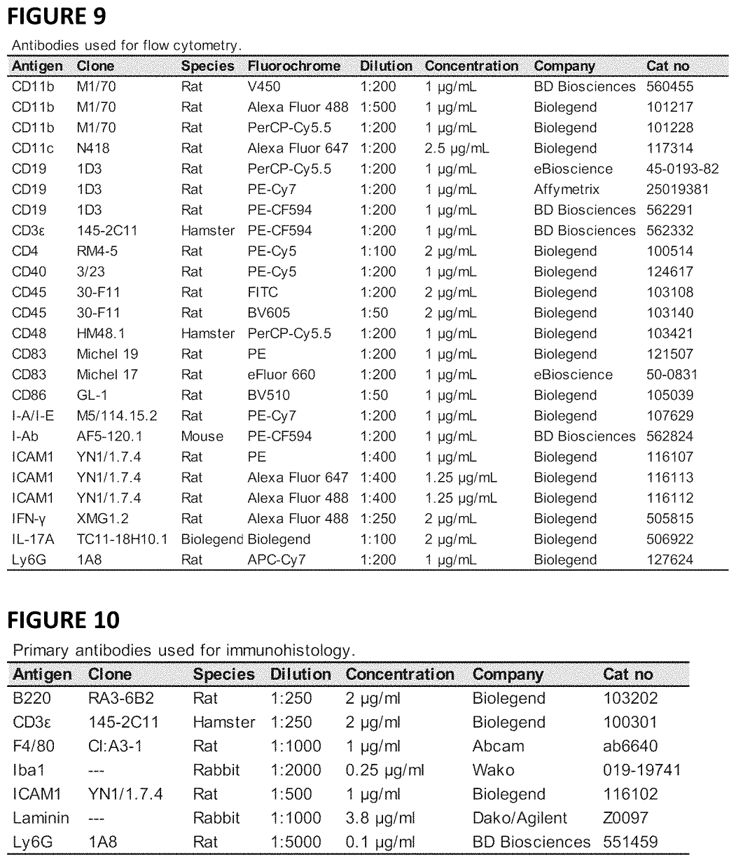

Blood samples were harvested by cardiac puncture in EDTA-treated cuvettes and treated with ammonium chloride solution (Stemcell Technologies) to remove erythrocytes. Mice were then anesthetized and exsanguinated by cardiac perfusion with saline. Spinal cords and lymph nodes were harvested, minced with razor blades in Dulbecco's PBS, digested for 45 min at 37° C. with 0.13 U/ml Liberase™ (Roche Diagnostics) and 50 U/ml DNase (Sigma-Aldrich) in Dulbecco's PBS, filtered through 40-μm cell strainers, then separated from myelin debris by centrifugation at 1,000×g in 35% Percoll (GE Healthcare). For immunostaining, cells were incubated on ice for 5 min with rat anti-CD16/CD32 antibody (BD Biosciences, clone 2.4G2, 5 μg/ml) and Fixable Viability Dye eFluor 506 or 455UV (eBioscience, 1:1000), then for 30 min with combinations of primary antibodies (FIG. 9). Cells were washed and resuspended in PBS before being analyzed/sorted with a FACSAria II flow cytometer (BD Biosciences). All analyses were performed by excluding dead cells and doublets using FlowJo (Tree Star). Gates were based on fluorescence-minus-one controls.

Microarray Analysis

Total RNA was extracted from FACS-purified cells using the miRNeasy Micro Kit (Qiagen). RNA (75 ng) was used to produce biotinylated single-strand cDNAs using the Affymetrix GeneChip WT Plus Reagent Kit. The cDNAs were hybridized to Affymetrix Mouse Gene 2.0 ST arrays for 16 h at 45° C. with constant rotation at 60 rpm. The arrays were washed and stained using the Affymetrix GeneChip Fluidics Station 450, then read using the Affymetrix GeneChip Scanner 3000 7G. Data were processed using the RMA algorithm in Affymetrix Expression Console and filtered in Microsoft Excel. Using the MeV software (TIGR), filtered data (intensity >100 in at least one group; standard deviation >100) were log₂-transformed and analyzed by hierarchical clustering using Spearman correlation. KEGG and Ingenuity Pathway Analysis were used to identify enriched pathways.

RT-qPCR

Total RNA was extracted from cells, spinal cord and blood using EZ-10 Spin Column Animal Total RNA Mini-preps Kit (Bio Basic), TRI-reagent (Sigma-Aldrich) and RNeasy Protect Animal Blood Kit (Qiagen), respectively. First strand cDNA was generated from 1-5 μg of RNA using Superscript III (Invitrogen) with random hexamers and 20-mer oligo-dT primers, then purified using the GenElute PCR Clean-Up Kit (Sigma-Aldrich). The product (20 ng) was analyzed using the LightCycler 480 system with the SYBR Green I Master mix and primers listed in Table 2 of the Sequence Listing according to the manufacturer's instructions (Applied Biosystems). The PCR conditions consisted of 45 cycles of denaturation (10 s at 95° C.), annealing (10 s at 60° C.), elongation (14 s at 72° C.) and reading (5 s at 74° C.). The number of mRNA copies was determined using the second derivative method⁶⁴.

Western Blotting

Bone marrow cells were collected by flushing out femurs with 10 mL of HBSS (Wisent Bioproducts), filtered through 70-μm mesh, resuspended in HBSS containing 45% Percoll (GE Healthcare), and centrifuged at 1,600×g for 30 min over a four-level Percoll density gradient (45%, 55%, 65%, 81%). Cell layers (mononuclear cells from the top and 45-55% interface; neutrophils from the 65%-81% interface) were retrieved and lysed by sonication (3×5 sec) in 50 μl RIPA buffer (50 mM Tris-HCl, 150 mM NaCl, 1% Triton X-100, 0.5% sodium deoxycholate, 0.1% sodium dodecyl sulfate (SDS), 1× protease and phosphatase inhibitor cocktails [Sigma-Aldrich, nos. P8340 and P5726], pH 8.0). Protein samples (20 μg) were boiled for 5 min in Laemmli buffer (50 mM Tris-HCl, 2% SDS, 6% β-mercaptoethanol, 10% glycerol, 12.5 μg/mL bromophenol blue), resolved on 15% SDS-polyacrylamide gel (Bio-Rad Mini-Protean II), and transferred to a polyvinylidene difluoride membrane for 45 min at 4° C. and 100 V in transfer buffer (25 mM Tris, 200 mM glycine) containing 20% methanol. The membrane was blocked for 30 min at room temperature in TSM (10 mM Tris, 150 mM NaCl, 5% non-fat dry milk, 0.1% Tween 20, pH 7.4), and incubated for 1 h at room temperature and then overnight at 4° C. in 7 mL of TSM containing polyclonal rabbit antibody to ASPRV1 C-terminus⁹ (1:1000). The membrane was washed four times in TSM for 5 min and then incubated for 1 h at room temperature with horseradish peroxidase-conjugated goat anti-rabbit antibody (1:2500; Jackson ImmunoResearch). The membrane was again washed four times in TSM, then twice in TS (10 mM Tris, 150 mM NaCl, pH 7.4) and once briefly in water, before being incubated for 60 sec with 1 mL Western Lightning chemiluminescence substrate (PerkinElmer), then blotted dry. The chemiluminescent signal was captured on Carestream Bio-Max Light Film (Kodak), which was scanned at 800 dpi and adjusted for contrast and brightness with Photoshop (Adobe). To control for protein loading, the membrane was rinsed overnight in TS and blotted for β-actin by 1-h incubations at room temperature first in mouse anti-β-actin antibody (1:5000; Millipore, clone C4), then in goat anti-mouse antibody (1:2500; Jackson ImmunoResearch).

Histology

Mice were transcardially perfused with saline, followed by ice-cold 4% paraformaldehyde in phosphate buffer, pH 7.4, over 10 min. Spinal cords were removed, postfixed for 4 h at 4° C., then cryoprotected overnight in 50 mM potassium phosphate-buffered saline supplemented with 20% sucrose. Series of sections were cut at 14 μm using a cryostat and stored at −20° C. until analysis. Immunofluorescence was performed, as described previously⁶⁵, using combinations of primary antibodies (FIG. 10). Slides were counterstained for 1 min with 2 μg/ml DAPI, then mounted with coverslips no. 1.5H and ProLong Diamond (Molecular Probes; refractive index, 1.47).

Confocal and STED Microscopy

Confocal images were acquired with a Leica TCS SP8 STED 3× microscope (equipped with white-light laser, 405-nm diode laser and HyD detectors) by sequential scanning using the following settings: objective, HC/PL/APO 63x/1.40 oil or 10×/0.4 dry; immersion oil, Leica Type F (refractive index, 1.518); scan speed, 600 Hz; line average, 2-4; time gate, 0.3-6.0 ns. Laser power and gain were set to optimize signal-to-noise ratio and avoid saturation using the QLUT Glow mode. Sizes of pixel, pinhole and z-step were set to optimize resolution or to oversample in the case of images to be deconvolved. STED was performed with a 775-nm depletion laser and the following adapted settings: objective, PL/APO 100×/1.40 oil STED White; line average, 6-8; time gate, 0.5-6.0 ns; STED 3D, 50-75%; depletion laser intensity, 60% (Alexa Fluor 594) or 30% (Atto 647N). Deconvolution was performed with Huygens Professional (Scientific Volume Imaging) using a theoretical point spread function, manual settings for background intensity and default signal-to-noise ratio. 3D reconstruction was performed with LAS X 3D Visualisation (Leica). Color balance, contrast and brightness were adjusted with Photoshop (Adobe).

Statistics

Unless otherwise indicated, data are expressed as mean±standard error. In general, means were compared with non-parametric (Wilcoxon, Kruskal-Wallis) or parametric tests (Student's t test, ANOVA) when data were continuous, normally distributed (Shapiro-Wilk W test) and of equal variance (Levene's test). EAE incidence curves were constructed using the Kaplan-Meier method and compared by Wilcoxon test. EAE severity curves were compared by two-way ANOVA with repeated measures using rank-transformed scores, followed by Wilcoxon test for pairwise comparisons using untransformed scores. Correlation between variables was determined using the Spearman's or Pearson's test. All these analyses were performed with JMP (SAS Institute) using a significance level of 5%.

### REFERENCES

1. Stromnes, I. M. & Goverman, J. M. Active induction of experimental allergic encephalomyelitis. Nat Protoc 1, 1810-1819 (2006).
2. Korn, T. & Kallies, A. T cell responses in the central nervous system. Nat Rev Immunol 17, 179-194(2017).
3. Rossi, B. & Constantin, G. Live Imaging of Immune Responses in Experimental Models of Multiple Sclerosis. Front Immunol 7, 506 (2016).
4. Mishra, M. K. & Yong, V. W. Myeloid cells—targets of medication in multiple sclerosis. Nat Rev Neurol 12, 593-551 (2016).
5. Kleinewietfeld, M. & Hafler, D. A. Regulatory T cells in autoimmune neuroinflammation. Immunol Rev 259, 231-244 (2014).
6. Casserly, C. S., Nantes, J. C., Whittaker Hawkins, R. F. & Vallieres, L. Neutrophil perversion in demyelinating autoimmune diseases: Mechanisms to medicine. Autoimmun Rev 16, 294-307 (2017).
7. Kostic, M. et al. IL-17 and glutamate excitotoxicity in the pathogenesis of multiple sclerosis. Scand J Immunol 79, 181-186 (2014).
8. Bernard, D. et al. Identification and characterization of a novel retroviral-like aspartic protease specifically expressed in human epidermis. J Invest Dermatol 125, 278-287 (2005).
9. Matsui, T. et al. Mouse homologue of skin-specific retroviral-like aspartic protease involved in wrinkle formation. J Biol Chem 281, 27512-27525 (2006).
10. Rhiemeier, V. et al. A novel aspartic proteinase-like gene expressed in stratified epithelia and squamous cell carcinoma of the skin. Am J Pathol 168, 1354-1364 (2006).
11. Hildenbrand, M. et al. Impaired skin regeneration and remodeling after cutaneous injury and chemically induced hyperplasia in taps-transgenic mice. J Invest Dermatol 130, 1922-1930(2010).
12. Matsui, T. et al. SASPase regulates stratum corneum hydration through profilaggrin-to-filaggrin processing. EMBO Mol Med 3, 320-333 (2011).
13. Michel, L. et al. B Cells in the Multiple Sclerosis Central Nervous System: Trafficking and Contribution to CNS-Compartmentalized Inflammation. Front Immunol 6, 636 (2015).
14. Weber, M. S. et al. B-cell activation influences T-cell polarization and outcome of anti-CD20 B-cell depletion in central nervous system autoimmunity. Ann Neurol 68, 369-383 (2010).
15. Wu, F., Cao, W., Yang, Y. & Liu, A. Extensive infiltration of neutrophils in the acute phase of experimental autoimmune encephalomyelitis in C57BL/6 mice. Histochem Cell Biol 133, 313-322 (2010).
16. Roy, M., Richard, J. F., Dumas, A. & Vallieres, L. CXCL1 can be regulated by IL-6 and promotes granulocyte adhesion to brain capillaries during bacterial toxin exposure and encephalomyelitis. J Neuroinflammation 9, 18 (2012).
17. Rumble, J. M. et al. Neutrophil-related factors as biomarkers in EAE and MS. J Exp Med 212, 23-35 (2015).
18. Richard, J. F., Roy, M., Audoy-Remus, J., Tremblay, P. & Vallieres, L. Crawling phagocytes recruited in the brain vasculature after pertussis toxin exposure through IL6, ICAM1, and ITGaM. Brain Pathol 21, 661-671 (2011).
19. Dumas, A. et al. The inflammasome pyrin contributes to pertussis toxin-induced IL-1beta synthesis, neutrophil intravascular crawling and autoimmune encephalomyelitis. PLoS Pathog.10, e1004150(2014).
20. Hasenberg, A. et al. Catchup: a mouse model for imaging-based tracking and modulation of neutrophil granulocytes. Nat Methods 12, 445-452 (2015).
21. Madisen, L. et al. A robust and high-throughput Cre reporting and characterization system for the whole mouse brain. Nat Neurosci 13, 133-140 (2010).
22. Audoy-Remus, J. et al. Rod-Shaped monocytes patrol the brain vasculature and give rise to perivascular macrophages under the influence of proinflammatory cytokines and angiopoietin-2. J. Neurosci. 28, 10187-10199 (2008).
23. Kishimoto, T. K., Jutila, M. A., Berg, E. L. & Butcher, E. C. Neutrophil Mac-1 and MEL-14 adhesion proteins inversely regulated by chemotactic factors. Science 245, 1238-1241 (1989).
24. Lacal, P., Pulido, R., SAnchez-Madrid, F. & Mollinedo, F. Intracellular location of T200 and Mol glycoproteins in human neutrophils. J Biol Chem 263, 9946-9951 (1988).
25. Bozoyan, L., Dumas, A., Patenaude, A. & Vallieres, L. Interleukin-36y is expressed by neutrophils and can activate microglia, but has no role in experimental autoimmune encephalomyelitis. J Neuroinflammation 12, 173 (2015).
26. Köffel, R. et al. Monocytic cell differentiation from band-stage neutrophils under inflammatory conditions via MKK6 activation. Blood 124, 2713-2724 (2014).
27. Ohsawa, K., Imai, Y., Kanazawa, H., Sasaki, Y. & Kohsaka, S. Involvement of Iba1 in membrane ruffling and phagocytosis of macrophages/microglia. J Cell Sci 113, 3073-3084 (2000).
28. Neumann, J. et al. Very-late-antigen-4 (VLA-4)-mediated brain invasion by neutrophils leads to interactions with microglia, increased ischemic injury and impaired behavior in experimental stroke. Acta Neuropathol 129, 259-277 (2015).
29. Friedl, P., den Boer, A. T. & Gunzer, M. Tuning immune responses: diversity and adaptation of the immunological synapse. Nat Rev Immunol 5, 532-545 (2005).
30. Barcia, C. et al. In vivo mature immunological synapses forming SMACs mediate clearance of virally infected astrocytes from the brain. J Exp Med 203, 2095-2107 (2006).
31. Barcia, C. et al. T cells' immunological synapses induce polarization of brain astrocytes in vivo and in vitro: a novel astrocyte response mechanism to cellular injury. PLoS One 3, e2977(2008).
32. Hashimoto, K., Joshi, S. K. & Koni, P. A. A conditional null allele of the major histocompatibility IA-beta chain gene. Genesis 32, 152-153 (2002).
33. Hjelmström, P., Juedes, A. E., Fjell, J. & Ruddle, N. H. B-cell-deficient mice develop experimental allergic encephalomyelitis with demyelination after myelin oligodendrocyte glycoprotein sensitization. J Immunol 161, 4480-4483 (1998).
34. Steinbach, K., Piedavent, M., Bauer, S., Neumann, J. T. & Friese, M. A. Neutrophils amplify autoimmune central nervous system infiltrates by maturing local APCs. J Immunol 191, 4531-4539(2013).
35. Yao, Y. et al. Neutrophil Priming Occurs in a Sequential Manner and Can Be Visualized in Living Animals by Monitoring IL-1beta Promoter Activation. J Immunol 194, 1211-1224 (2015).
36. Woodfin, A. et al. ICAM-1-expressing neutrophils exhibit enhanced effector functions in murine models of endotoxemia. Blood 127, 898-907 (2016).
37. Heemskerk, N., Asimuddin, M., Oort, C., van Rijssel, J. & van Buul, J. D. Annexin A2 Limits Neutrophil Transendothelial Migration by Organizing the Spatial Distribution of ICAM-1. J Immunol 196, 2767-2778 (2016).
38. Pillay, J. et al. A subset of neutrophils in human systemic inflammation inhibits T cell responses through Mac-1. J Clin Invest 122, 327-336 (2012).
39. Vono, M. et al. Neutrophils acquire the capacity for antigen presentation to memory CD4(+) T cells in vitro and ex vivo. Blood 129, 1991-2001 (2017).
40. Okuda, K., Tani, K., Ishigatsubo, Y., Yokota, S. & David, C. S. Antigen-pulsed neutrophils bearing la antigens can induce T lymphocyte proliferative response to the syngeneic or semisyngeneic antigen-primed T lymphocytes. Transplantation 30, 368-372 (1980).
41. Culshaw, S., Millington, O. R., Brewer, J. M. & McInnes, I. B. Murine neutrophils present Class II restricted antigen. Immunol Lett 118, 49-54 (2008).
42. Abi Abdallah, D. S., Egan, C. E., Butcher, B. A. & Denkers, E. Y. Mouse neutrophils are professional antigen-presenting cells programmed to instruct Thi and Tₕ17 T-cell differentiation. Int Immunol 23, 317-326 (2011).
43. Ostanin, D. V. et al. Acquisition of antigen-presenting functions by neutrophils isolated from mice with chronic colitis. J Immunol 188, 1491-1502 (2012).
44. Strasser, B., Mlitz, V., Fischer, H., Tschachler, E. & Eckhart, L. Comparative genomics reveals conservation of filaggrin and loss of caspase-14 in dolphins. Exp Dermatol 24, 365-369 (2015).
45. Li, R. et al. Cytokine-Defined B Cell Responses as Therapeutic Targets in Multiple Sclerosis. Front Immunol 6, 626 (2015).
46. Oliver, A. R., Lyon, G. M. & Ruddle, N. H. Rat and human myelin oligodendrocyte glycoproteins induce experimental autoimmune encephalomyelitis by different mechanisms in C57BL/6 mice. J Immunol 171, 462-468 (2003).
47. Wolf, S. D., Dittel, B. N., Hardardottir, F. & Janeway, C. A. Experimental autoimmune encephalomyelitis induction in genetically B cell-deficient mice. J Exp Med 184, 2271-2278 (1996).
48. Dittel, B. N., Urbania, T. H. & Janeway, C. A. Relapsing and remitting experimental autoimmune encephalomyelitis in B cell deficient mice. J Autoimmun 14, 311-318 (2000).
49. Fillatreau, S., Sweenie, C. H., McGeachy, M. J., Gray, D. & Anderton, S. M. B cells regulate autoimmunity by provision of IL-10. Nat Immunol 3, 944-950 (2002).
50. Lyons, J. A., Ramsbottom, M. J., Mikesell, R. J. & Cross, A. H. B cells limit epitope spreading and reduce severity of EAE induced with PLP peptide in BALB/c mice. J Autoimmun 31, 149-155 (2008).
51. Lehmann-Horn, K. et al. Anti-CD20 B-cell depletion enhances monocyte reactivity in neuroimmunological disorders. J Neuroinflammation 8, 146 (2011).
52. Matsushita, T. et al. Inhibitory role of CD19 in the progression of experimental autoimmune encephalomyelitis by regulating cytokine response. Am J Pathol 168, 812-821 (2006).
53. Matsushita, T., Yanaba, K., Bouaziz, J. D., Fujimoto, M. & Tedder, T. F. Regulatory B cells inhibit EAE initiation in mice while other B cells promote disease progression. J Clin Invest 118, 3420-3430(2008).
54. Matsushita, T., Horikawa, M., Iwata, Y. & Tedder, T. F. Regulatory B cells (B10 cells) and regulatory T cells have independent roles in controlling experimental autoimmune encephalomyelitis initiation and late-phase immunopathogenesis. J Immunol 185, 2240-2252(2010).
55. Ray, A., Mann, M. K., Basu, S. & Dittel, B. N. A case for regulatory B cells in controlling the severity of autoimmune-mediated inflammation in experimental autoimmune encephalomyelitis and multiple sclerosis. J Neuroimmunol 230, 1-9 (2011).
56. Ray, A., Basu, S., Williams, C. B., Salzman, N. H. & Dittel, B. N. A novel IL-10-independent regulatory role for B cells in suppressing autoimmunity by maintenance of regulatory T cells via GITR ligand. J Immunol 188, 3188-3198 (2012).
57. Larochelle, C. et al. Immunological and pathological characterization of fatal rebound MS activity following natalizumab withdrawal. Mult Scler 23, 72-81 (2017).
58. Reynolds, R. et al. The neuropathological basis of clinical progression in multiple sclerosis. Acta Neuropathol 122, 155-170 (2011).
59. Rachel, R. A. et al. Melanoregulin, product of the dsu locus, links the BLOC-pathway and OA1 in organelle biogenesis. PLoS One 7, e42446 (2012).
60. Maruyama, M., Lam, K. P. & Rajewsky, K. Memory B-cell persistence is independent of persisting immunizing antigen. Nature 407, 636-642 (2000).
61. Chen, J. et al. Immunoglobulin gene rearrangement in B cell deficient mice generated by targeted deletion of the JH locus. Int Immunol 5, 647-656 (1993).
62. Dang, A. K., Jain, R. W., Craig, H. C. & Kerfoot, S. M. B cell recognition of myelin oligodendrocyte glycoprotein autoantigen depends on immunization with protein rather than short peptide, while B cell invasion of the CNS in autoimmunity does not. J Neuroimmunol 278, 73-84 (2015).
63. Jain, R. W., Dang, A. K. & Kerfoot, S. M. Simple and Efficient Production and Purification of Mouse Myelin Oligodendrocyte Glycoprotein for Experimental Autoimmune Encephalomyelitis Studies. J Vis Exp (2016).
64. Luu-The, V., Paquet, N., Calvo, E. & Cumps, J. Improved real-time RT-PCR method for high-throughput measurements using second derivative calculation and double correction. Biotechniques 38, 287-293 (2005).
65. Vallieres, L. & Sawchenko, P. E. Bone marrow-derived cells that populate the adult mouse brain preserve their hematopoietic identity. J Neurosci 23, 5197-5207 (2003).
66. Hawkins, R. F. W., A. Patenaude, A. Dumas, R. Jain, Y. Tesfagiorgis, S. Kerfoot, T. Matsui, M. Gunzer, P. E. Poubelle, C. Larochelle, M. Pelletier, and L. Vallieres. 2017. ICAM1+ neutrophils promote chronic inflammation via ASPRV1 in B cell-dependent autoimmune encephalomyelitis. JCI Insight. 2:96882. doi:10.1172/jci.insight.96882.

### SEQUENCES

SEQ ID NO.: 1
>NM_026414.2 Mus musculus aspartic peptidase,
retroviral-like 1 (Asprv1), mRNA
ATGAGGAACCCTGGGGGCCCAGGTTGGGCATCAAAAAGGCCCCTGCAGAA
GAAGCAGAACACAGCCTGCCTCTGTGCCCAGCAGCCAGCCAGACACTTTG
TACCGGCTCCCTTCAACTCGTCCAGGCAGGGCAAGAACACGGCCCAGCCG
ACAGAGCCCTCGCTCTCCAGCGTGATTGCGCCCACACTCTTCTGTGCGTT
TCTTTACTTGGCTTGTGTTACTGCTGAACTTCCAGAGGTGAGCAGAAGGA
TGGCCACCAGCGGAGTCAGAAGCAAGGAAGGACGCCGGGAGCATGCCTTC
GTCCCAGAACCTTTCACTGGTACTAACTTAGCTCCCAGCCTTTGGCTGCA
CCGCTTTGAAGTCATTGATGACCTCAACCATTGGGATCATGCCACCAAAC
TGAGGTTCCTGAAAGAGTCGCTCAAGGGAGATGCCCTGGATGTCTACAAT
GGACTCAGTTCCCAGGCCCAGGGCGATTTCAGTTTTGTGAAGCAAGCCCT
CCTGAGGGCCTTTGGGGCCCCTGGGGAGGCCTTCAGTGAGCCCGAAGAGA
TTTTGTTTGCCAACAGCATGGGTAAGGGCTACTACCTTAAAGGGAAGGTT
GGCCATGTGCCTGTGAGATTCCTGGTGGACTCTGGGGCTCAGGTGTCTGT
GGTTCACCCCGCCTTATGGGAGGAGGTCACTGATGGTGACCTGGATACTC
TTCGTCCTTTTAACAATGTGGTCAAAGTGGCCAATGGGGCAGAGATGAAG
ATCTTGGGTGTGTGGGACACAGAAATTAGCCTGGGCAAGACAAAGCTGAA
GGCCGAGTTTCTGGTGGCCAACGCCAGCGCAGAAGAGGCTATTATTGGCA
CAGACGTCTTGCAGGACCACAATGCCGTGCTGGACTTCGAACACCGCACC
TGCACCCTGAAGGGGAAGAAGTTCCGCCTGCTCCCTGTCGGGAGCTCCTT
GGAGGATGAGTTTGACCTGGAGCTTATTGAGGAAGAGGAGGGGTCTTCTG
CACCGGAGGGCTCCCACTAAGAAACCCCATTTCTTGTTCCCAGCATTGGT
AGGGGGACTTTGTGTTGGGGGGAGCAGATGTCCTGGGGGGTATCATCCGG
CCTAGCCAGTCTTTACACCGGTTCTCAGTTTCCCTCCTTCTACAGGGGCC
TTGCTTTGCCTTTGTTTGGGGAGGGAGGCCAGCTTGGTGGCCTAAAGCAG
TGTCCCCAAGGTCTGCAAAGACTTCCAAGGCTGGCAGGAGCTTCTGAGGA
AGCCAGGAATGTCAATCTTGAGAGAGGACCCTTTTAGATCCCCTGAAGTA
TGGCTCAGTCACTTTCACGTCCCCAAGCCTGCTGAGCTGAGCCTGGTCTT
GGCTAAGACCCTCACAATCCAGATGCTTGGAGGAGACTGGCAGCTGCTCT
GGGAGTCCTCCCTGAGTCCTCCCACCTGCACAAGGATGCTCCCTCCTGTC
CTGTCACTTGCCTTGAATCTCATGGAGCCTGTATCAATAATTCAATTATT
TCAAAACACCAATAAAGATCTGTTCATGG

The cDNA encoding SEQ ID NO.2 corresponds to nucleotide sequences 1-1020 of SEQ ID NO.:1

>NP_080690 length = 339 MW = 37174.1 Murine ASPRV1
SEQ ID NO.: 2
MRNPGGPGWASKRPLQKKQNTACLCAQQPARHFVPAPFNSSRQGKNTAQP

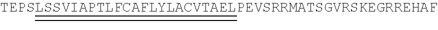

VPEPFTGTNLAPSLWLHRFEVIDDLNHWDHATKLRFLKESLKGDALDVYN
GLSSQAQGDFSFVKQALLRAFGAPGEAFSEPEEILFANSMGKGYYLKGKV
GHVPVRFLVDSGAQVSVVHPALWEEVTDGDLDTLRPFNNVVKVANGAEMK
ILGVWDTEISLGKTKLKAEFLVANASAEEAIIGTDVLQDHNAVLDFEHRT
CTLKGKKFRLLPVGSSLEDEFDLELIEEEEGSSAPEGSH
Legend:
Active site underlined
Key aspartic acid residue in bold (D210)

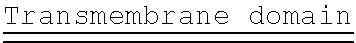

SEQ ID NO.: 3
>NM_152792.2 Homo sapiens aspartic peptidase
retroviral like 1 (ASPRV1), mRNA
GCAGCAGTTGAGTCCATGTAAGATGATTGTGCACGTGTGTGTGTGTGTGT
GTGTGTGTGTGTGTGTCCTCACTCACTTAAAAAAGACCATTTCTGTGCAT
TCTTTATGCTGGAACCCTCCTCTGCCTCCCTCTCCAGGAAATCCCATAGG
ATCCCATACCTGCTTTAGAATGTGTGTGAGACACTTCCCAGTGCCTGGCA
TGTGGTAGGAGCTCAGTACATGTAAACTGTTTTCCTTTGACCACTCAAAG
TAGTCACTTGTTGGGGAGGCCAATGGCGGCTTCCCCGGTCTCTGAGCTGA
TGGGCGTCTGGTCCCTGCCTCCCCTTCCTTCACTGGCTGATGACAAATAC
CTGAATGTCATCACATCCTCCGTGTCCTCATCTGTTATTCTGGAACACAG
GCCAGGGAGGGTACAGGGTAGTCCCCCTTCAACAGAGGCTGGCCTTTCTT
GGGATGTGGGCTGGCTGGTGTTGGTCCAAGGATGGATGCAGAGGGTGAGG
CACCCATCCTGCTAGTCCGGCCGGATGCTGGCAGGAGGGCGGGGTGAGGA
GGGGCGGAGCTTCCAGAACAAAGGAGAATGGGGAGCCCAGGGGCCAGCCT
AGGCATCAAAAAGGCTCTGCAGAGTGAACAGGCCACAGCACTGCCTGCCT
CTGCCCCAGCAGTCAGCCAGCCGACCGCGCCTGCTCCCTCCTGCTTGCCC
AAGGCCGGGCAAGTCATCCCCACTCTGCTTCGAGAGGCCCCGTTTTCCAG
CGTGATTGCGCCGACACTGCTCTGTGGGTTTCTCTTCTTGGCGTGGGTTG
CTGCTGAGGTTCCAGAGGAGAGCAGCAGGATGGCCGGGAGCGGAGCCAGG
AGTGAGGAAGGCCGCCGGCAGCATGCCTTCGTCCCGGAACCTTTTGATGG
GGCCAATGTCGTCCCAAACCTCTGGCTGCACAGCTTTGAAGTCATCAATG
ACCTCAACCATTGGGACCATATCACCAAGCTAAGGTTCCTGAAAGAGTCC
CTCAGAGGAGAGGCCCTGGGTGTCTACAATAGGCTCAGTCCCCAGGACCA
GGGAGACTATGGGACTGTGAAAGAGGCCCTCCTGAAGGCCTTTGGGGTCC
CTGGGGCTGCCCCCAGCCACCTGCCCAAAGAGATCGTCTTTGCCAACAGC
ATGGGTAAGGGCTACTATCTCAAGGGGAAGATTGGCAAAGTGCCCGTGAG
GTTCCTGGTGGACTCTGGGGCCCAGGTCTCTGTGGTCCACCCAAACTTGT
GGGAGGAGGTCACTGATGGCGATCTGGACACCCTGCAGCCCTTTGAGAAT
GTGGTAAAGGTGGCCAATGGTGCTGAAATGAAGATCCTGGGTGTCTGGGA
TACAGCGGTGTCCCTAGGCAAGCTGAAGCTGAAGGCACAGTTCCTAGTGG
CCAATGCGAGTGCCGAGGAAGCCATCATTGGCACTGATGTGCTCCAGGAC
CACAATGCTATCCTGGACTTTGAGCACCGCACATGCACCCTGAAAGGGAA
GAAGTTTCGCCTTCTGCCTGTGGGAGGGTCCCTGGAAGATGAGTTTGACC
TGGAGCTCATAGAGGAGGACCCCTCCTCAGAAGAAGGGCGGCAGGAGCTA
TCCCACTGAGAAGCCACCTTTTCTTTAACCTCCTAAATATTGGTGGGAAG
ACCCACCGCTGTGGGGGGGGTTGCATATCCTCATGGGGGTCACTGGGCTT
GGCCAGTCTGCTTATCAACTCTTGCTCTTCTCTCCCCTTTGCCTCCCTCT
GCAGGGGCCTTAATCTGCCCCTGGTAGGGGAGGCTTCCACTGAACAGGCA
CAGGTGAGGGAGAGCAGGCTGGCTTAGAGGGACAGGGTCCCCATGGTCAT
CAAGCTGCTGTTGATGACAAAGACTCAAAGGCTGGAAGAGCTCCCAAGGA
AGCTAGAAATGCTTGTCTTTGAAAGAACTGTGGGACCCCTTCAGATTCCC
TGAGGTATGGCTTGGTCACTCTCAGGTCCTCAAAGCCTGTCTTAGTTGGG
CTGGGTCCTAGCTGCAGGGTCTTTGTGAGGGTCACAGTTGCTCTGGGACA
CCTCCCTGAAGAGCCTTTCCACCTGTACAATCGTATTTTCTTTCTGTCAT
TTGCTTTGAAGCCCATTGTGCCTTATGCCAATAATCAATTGCTGCAAACA
CCAATAAAGATTGATTCATGGAAAAAAAAAAAAAAAAAAAAAAAAAAAAA
AAAAAAAAAAAAAAAA

The cDNA encoding SEQ ID NO.4 corresponds to nucleotide sequences 578 to 1609 of SEQ ID NO.:3

>NP_690005 length = 343 MW = 36991.1 Human ASPRV1
SEQ ID NO.: 4
MGSPGASLGIKKALQSEQATALPASAPAVSQPTAPAPSCLPKAGQVIPTL

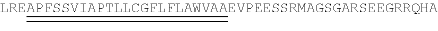

FVPEPFDGANVVPNLWLHSFEVINDLNHWDHITKLRFLKESLRGEALGVY
NRLSPQDQGDYGTVKEALLKAFGVPGAAPSHLPKEIVFANSMGKGYYLKG
KIGKVPVRFLVDSGAQVSVVHPNLWEEVTDGDLDTLQPFENVVKVANGAE
MKILGVWDTAVSLGKLKLKAQFLVANASAEEAIIGTDVLQDHNAILDFEH
RTCTLKGKKFRLLPVGGSLEDEFDLELIEEDPSSEEGRQELSH
Legend:
Active site underlined
Key aspartic acid residue in bold (D212)

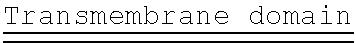

SEQ ID NO.: 5
short form of mouse ASPRV1
MATSGVRSKEGRREHAFVPEPFTGTNLAPSLWLHRFEVIDDLNHWDHATK
LRFLKESLKGDALDVYNGLSSQAQGDFSFVKQALLRAFGAPGEAFSEPEE
ILFANSMGKGYYLKGKVGHVPVRFLVDSGAQVSVVHPALWEEVTDGDLDT
LRPFNNVVKVANGAEMKILGVWDTEISLGKTKLKAEFLVANASAEEAIIG
TDVLQDHNAVLDFEHRTCTLKGKKFRLLPVGSSLEDEFDLELIEEEEGSS
APEGSH
SEQ ID NO.: 6
short form of human ASPRV1
MAGSGARSEEGRRQHAFVPEPFDGANVVPNLWLHSFEVINDLNHWDHITK
LRFLKESLRGEALGVYNRLSPQDQGDYGTVKEALLKAFGVPGAAPSHLPK
EIVFANSMGKGYYLKGKIGKVPVRFLVDSGAQVSVVHPNLWEEVTDGDLD
TLQPFENVVKVANGAEMKILGVWDTAVSLGKLKLKAQFLVANASAEEAII
GTDVLQDHNAILDFEHRTCTLKGKKFRLLPVGGSLEDEFDLELIEEDPSS
EEGRQELSH

| Query = human ASPRV1 | | | |
|---|---|---|---|
| Sbjct = mouse ASPRV1 | | | |
| NW Score Identities Positives Gaps | | | |
| 1190 248/345(72%) 271/345(78%) 8/345(2%) | | | |
| Query | 1 | MGSPGA-SLGIKKALQSEQATA-LPASAPAVSQPTAPAPSCLPKAGQVIPTLLREAPFSS | 58 |
| | | M +PG K+ LQ +Q TA L A PA AP S PT E SS | |
| Sbjct | 1 | MRNPGGPGWASKRPLQKKQNTACLCAQQPARHFVPAPFNSSRQGKNTAQPT---EPSLSS | 57 |
| Query | 59 | VIAPTLLCGFLFLAWVAAEVPEESSRMAGSGARSEEGPRQHAFVPEPFDGANVVPNLWLH | 118 |
| | | VIAPTL C FL+LA V AE+PE S RMA SG RS+EGRR+HAFVPEPF G N+ P+LWLH | |
| Sbjct | 58 | VIAPTLFCAFLYLACVTAELPEVSRRMATSGVRSKEGRREHAFVPEPFTGTNLAPSLWLH | 117 |
| Query | 119 | SFEVINDLNHWDHITKLRFLKESLRGEALGVYNRLSPQDQGDYGTVKEALLKAFGVPGAA | 178 |
| | | FEVI+DLNHWDH TKLRFLKESL+G+AL VYN LS Q QGD+ VK+ALL+AFG PG A | |
| Sbjct | 116 | RFEVIDDLNHWDHATKLRFLKESLKGDALDVYNGLSSQAQGDFSFVKQALLRAFGAPGEA | 177 |
| Query | 179 | PSHLPKEIVFANSMGKGYYLKGKIGKVPVRFLVDSGAQVSVVHPNLWEEVTDGDLDTLQP | 236 |
| | | S P+EI+FANSMGKGYYLKGK+G VPVRFLVDSGAQVSVVHP LWEEVTDGDLDTL+P | |
| Sbjct | 178 | FSE-PEEILFANSMGKGYYLKGKVGHVPVRFLVDSGAQVSVVHPALWEEVTDGDLDTLRP | 236 |
| Query | 239 | FENVVKVANGAEMKILGVWDTAVSLGKLKLKAQFLVANASAEEAIIGTDVLQDHNAILDF | 298 |
| | | F NVVKVANGAEMKILGVWDT +SLGK KLKA+FLVANASAEEAIIGTDVLQDHNA+LDF | |
| Sbjct | 237 | FNNVVKVANGAEMKILGVWDTEISLGKTKLKAEFLVANASAEEAIIGTDVLQDHNAVLDF | 296 |
| Query | 299 | EHRTCTLKGKKFRLLPVGGSLEDEFDLELIEEDPSSEEGRQELSH | 343 |
| | | EHRTCTLKGKKFRLLPVG SLEDEFDLELIEE+ S E SH | |
| Shirt | 297 | ERPTCTLKGKKFRLLPVGSSLEDEFDLELIEEKKGSSA--PEGSH | 339 |

TABLE 1

| | Primers | | |
|---|---|---|---|
| Target | Forward | Reverse | RefSeq |
| ASPRV1, human | 5′-ccacctgcccaaagagatcgt-3′ | 5′-atcgccatcagtgacctcctc-3′ | NM_152792.2 |
| cDNA | | | |
| ASPRV1, mouse | 5′-atgggaggaggtcactgatggt-3′ | 5′-ctcggccttcagctttgtcttg-3′ | NM_026414 |
| cDNA | | | |
| GAPDH, human cDNA | 5′-ggctctccagaacatcatccct-3′ | 5′-acgcctgcttcaccaccttctt-3′ | NM_002046 |
| H2-Ab1, mouse cDNA, | 5′-gactgccattacctgtgccttaga-3′ | 5′-atgaactggtacacgaaatgcctt- | NM_207105 |
| exon 1-2 junction | | 3′ | |
| H2-Ab1, mouse cDNA, | 5′-ctccctgcggcggcttgaac-3′ | 5′- | NM_207105 |
| exon 2-3 junction | | cctaataagctgtgtggatgagaccc- | |
| | | 3′ | |
| H2-Ab1, | 5′-gactgccattacctgtgccttaga-3′ | 5′-ctcagcaccatcagcaccacc-3′ | NM_207105 |
| mouse gene, exon1 | | | |
| H2-Ab1, | 5′- | 5′-ggctcttcaggctgggatgct-3′ | NM_207105 |
| mouse gene, exon3 | gggtctcatccacacagcttattagg-3′ | | |
| HPRT1, | 5′- | 5′- | NM_013556 |
| mouse cDNA | caggactgaaagacttgctcgagat- | cagcaggtcagcaaagaacttatagc- | |
| | 3′ | 3′ | |

TABLE 2

| Mouse | | | |
|---|---|---|---|
| strain | Allele | Forward primer | Reverse primer |
| Ai6 | Mutated | 5′-ggcattaaagcagcgtatcc-3 | 5′-aaccagaagtggcacctgac-3′ |
| | Wild- | 5′-aagggagctgcagtggagta-3′ | 5′-ccgaaaatctgtgggaagtc-3′ |
| | type | | |
| ASPRV1−/− | Mutated | 5′-ccaagttctattccatcaga-3′ | 5′-actatctatctagctatctgcatgtctatc-3′ |
| | Wild- | 5′-gcttgagcccttgagcccacagatttac-3′ | 5′-actatctatctagctatctgcatgtctatc-3′ |
| | type | | |
| B1-8 | Mutated | 5′-gaggagacggtgaccgtggtccctgc-3′ | 5′-ggaccagggggctcaggtcactcagg-3′ |
| | Wild- | 5′-ctactggatgcactgggtga-3′ | 5′-ttggccccagtagtcaaagta-3′ |
| | type | | |
| Catchup | Mutated | 5′-acgtccagacacagcatagg-3′ | 5′-gaggtccaagagactttctgg-3′ |
| | Wild- | 5′-ggttttatctgtgcagccc-3′ | 5′-gaggtccaagagactttctgg-3′ |
| | type | | |
| H2-Ab1- | Mutated | 5′-ctctacacccccaacacacc-3′ | 5′-tcgccttcttgacgagttct-3′ |
| floxed | Wild- | 5′-ctctacacccccaacacacc-3′ | 5′-agtgagcgagcacagacaag-3′ |
| | type | | |
| IL-36γ−/− | Mutated | 5′-ggcggatttctgagttggag-3′ | 5′-gcagcgcatcgccttctatc-3′ |
| | Wild- | 5′-ctgggctatttgtatcttca-3′ | 5′-cacacctgctggtccaagtc-3′ |
| | type | | |
| MREG−/− | Mutated | 5′-ctgggagttcaaggttggtct-3′ | 5′-ccacagtctcaagtctttcct-3′ |
| | Wild- | 5′-ctgggagttcaaggttggtct-3′ | 5′-gcaggagagggctgggaaaaa-3′ |
| | type | | |

1. A method for identifying mammalian neutrophils, the method comprising detecting the aspartic peptidase retroviral-like 1 (ASPRV1) protein or an ASPRV1 fragment thereof, a nucleic acid encoding ASPRV1, a nucleic acid complement or a nucleic acid fragment thereof.

2. (canceled)

3. The method of claim 1, wherein the mammalian neutrophils are from a mouse, a human, or a primate.

4. The method of claim 1, wherein the ASPRV1 protein or fragment thereof is detected with an anti-ASPRV1 antibody or an antigen-binding fragment thereof or wherein the ASPRV1 protein or fragment thereof, or the nucleic acid encoding ASPRV1, the nucleic acid complement or the nucleic acid fragment thereof is detected by a method comprising an amplification and/or hybridization step.

5-7. (canceled)

8. The method of claim 1, wherein the method comprises obtaining a biological sample from the mammal and determining the presence of the ASPRV1 protein or a fragment thereof or of a nucleic acid encoding the ASPRV1 protein in the biological sample.

9. A method for the diagnosis or prognosis of a demyelinating disease in a mammal, the method comprising detecting neutrophils in the mammal and/or quantifying neutrophils in the mammal.

10-11. (canceled)

12. The method of claim 9, wherein the neutrophils are detected by determining the presence of the ASPRV1 protein or fragment thereof, or the nucleic acid encoding ASPRV1, the nucleic acid complement or the nucleic acid fragment thereof.

13. (canceled)

14. The method of claim 12, wherein the presence of the ASPRV1 protein or fragment thereof is determined with an antibody or an antigen-binding fragment thereof that specifically binds to ASPRV1 or wherein the presence of the ASPRV1 protein or fragment thereof, or the nucleic acid encoding ASPRV1, the nucleic acid complement or the nucleic acid fragment thereof is determined by a method comprising an amplification and/or a hybridization step.

15-16. (canceled)

17. A method of targeting neutrophils in a mammal, the method comprising administering to the mammal a compound that specifically binds to the ASPRV1 protein or a fragment thereof or to a nucleic acid encoding the ASPRV1 protein, a nucleic acid complement or a nucleic acid fragment thereof or that inhibits ASPRV1 expression or enzymatic activity.

18. (canceled)

19. The method of claim 17, wherein the compound is an antibody or an antigen-binding fragment thereof that specifically binds to the ASPRV1 protein or to a fragment thereof.

20. The method of claim 19, wherein the antibody or antigen-binding fragment thereof comprises:
a. an antibody or an antigen binding fragment that inhibits the dimerization of the ASPRV1 protein or of the fragment thereof,
b. antibody or antigen-binding fragment thereof that inhibits the enzymatic activity of the ASPRV1 protein or of the fragment thereof, or;
c. antibody or antigen-binding fragment thereof that inhibits auto-cleavage of the ASPRV1 protein or of the fragment thereof.

21-22. (canceled)

23. The method of claim 19, wherein the antibody or antigen-binding fragment thereof is a polyclonal antibody, a monoclonal antibody, a humanized antibody, a chimeric antibody, a human antibody or an antigen-binding fragment thereof.

24. The method of claim 19, wherein the antibody or antigen-binding fragment thereof is conjugated with a cargo moiety, wherein the cargo moiety optionally comprises a cytostatic agent or a cytotoxic agent.

25. (canceled)

26. A method of treating a mammal having an inflammatory or autoimmune disorder associated with neutrophils, the method comprising administering a compound that inhibits or sequester the ASPRV1 protein, an ASPRV1 fragment or a nucleic acid encoding the ASPRV1 protein.

27-28. (canceled)

29. The method of claim 26, wherein the compound is an antibody that specifically binds to the ASPRV1 protein or an antigen-binding fragment thereof.

30-32. (canceled)

33. The method of claim 26, wherein the autoimmune disorder associated with neutrophils is a demyelinating autoimmune disease.

34. The method of claim 33, wherein the demyelinating autoimmune disease is selected from the group consisting of multiple sclerosis, neuromyelitis optica spectrum disorder and acute disseminated encephalomyelitis.

35. The method of claim 26, wherein the compound is administered during the acute phase or chronic phase of the disorder.

36-45. (canceled)

46. An antibody or antigen binding fragment thereof that specifically binds to a short form of ASPRV1 having the amino acid sequence set forth in SEQ ID NO.:5 or in SEQ ID NO.:6.

47. A pharmaceutical composition comprising the antibody or antigen-binding fragment thereof of claim 46 and a pharmaceutically acceptable carrier.

48-55. (canceled)

56. A method of identifying an inhibitor of neutrophils, the method comprising contacting neutrophils with an antibody or antigen-binding fragment thereof that specifically binds to the ASPRV1 protein or to a fragment thereof and assessing the activity of the neutrophils.

57-58. (canceled)

59. The method of claim 56, wherein the activity of neutrophils is assessed by differential expression profile.

60-61. (canceled)

62. A method for treating a disease associated with neutrophils in a mammal in need thereof, the method comprising detecting ASPRV1 in a biological sample obtained from the mammal and administering a drug that modulates the activity of neutrophils or that reduces neutrophils number.

63. A method of treating a mammal having a disease associated with neutrophils, the method comprising administering an antibody or an antigen binding fragment thereof that specifically binds to the ASPRV1 protein or to a fragment thereof.

64. The method of claim 63, wherein the disease is an inflammatory or autoimmune disorder.

65. The method of claim 63, wherein the fragment thereof is an ASPRV1 active fragment thereof.

66. The method of claim 63, wherein the ASPRV1 protein is a short form of ASPRV1 having the amino acid sequence set forth in SEQ ID NO.:6.
